(19) United States
(12) Patent Application Publication
(10) Pub. No.: US 2025/0026744 A1
(43) Pub. Date: Jan. 23, 2025

(54) FXR (NR1H4) MODULATING COMPOUNDS

(71) Applicant: Gilead Sciences, Inc., Foster City, CA (US)

(72) Inventors: Peter A. Blomgren, Issaquah, WA (US); Kevin S. Currie, North Bend, WA (US); Morin Mae Frick, San Mateo, CA (US); Elizabeth M. Horstman, San Mateo, CA (US); Joshua A. Kaplan, Acton, MA (US); Jeffrey E. Kropf, Issaquah, WA (US); William J. Watkins, Saratoga, CA (US)

(21) Appl. No.: 18/747,316

(22) Filed: Jun. 18, 2024

Related U.S. Application Data
(63) Continuation of application No. 17/534,951, filed on Nov. 24, 2021, now abandoned.
Application No. 17/534,951 is a continuation of application No. 16/741,101, filed on Jan. 13, 2020, now Pat. No. 11,225,473.
(60) Provisional application No. 62/792,714, filed on Jan. 15, 2019.

(51) Int. Cl. C07D 413/14; A61K 31/397; A61K 31/4439; A61K 31/519; A61K 31/53; A61P 1/16

(52) U.S. Cl. CPC C07D 413/14; A61P 1/16; A61K 31/397; A61K 31/4439; A61K 31/519; A61K 31/53; C07B 2200/13

(57) ABSTRACT

The present disclosure relates generally to compounds that bind to FXR and act as agonists of FXR. The disclosure further relates to the use of the compounds for the preparation of a medicament for the treatment of diseases and/or conditions through binding of said nuclear receptor by said compounds and to a process for the synthesis of said compounds.

### CROSS-REFERENCE TO RELATED APPLICATIONS

This application is a continuation of U.S. patent application Ser. No. 17/534,951, filed Nov. 24, 2021, which is a continuation of U.S. patent application Ser. No. 16/741,101, filed Jan. 13, 2020, now U.S. Pat. No. 11,225,473, which claims the benefit of priority to U.S. Provisional Patent Application No. 62/792,714, filed Jan. 15, 2019, titled “FXR (NR1H4) MODULATING COMPOUNDS,” the contents of which are hereby incorporated by reference in their entireties.

### REFERENCE TO AN ELECTRONIC SEQUENCE LISTING

The contents of the electronic sequence listing (37JD-313708.xml: Size: 2,252 bytes: and Date of Creation: Jun. 18, 2024) is herein incorporated by reference in its entirety.

### FIELD

The present disclosure relates to compounds that bind to and act as agonists or modulators of the Farnesoid X Receptor (FXR) and act as agonists or modulators of FXR. The disclosure further relates to the use of the compounds for the treatment and/or prophylaxis of diseases and/or conditions by said compounds.

### BACKGROUND

The Farnesoid X Receptor (FXR), also often referred to as NR1H4 (nuclear receptor subfamily 1, group H, member 4) when referring to the human receptor, is a nuclear hormone receptor. FXR has been associated with multiple biological functions. FXR is primarily expressed in the liver and throughout the entire gastrointestinal tract, but is also found in the kidney, adrenal glands, and ovary. FXR is associated with controlling intracellular gene expression, and may be involved in paracrine and endocrine signaling. In the intestine and liver, FXR functions as a regulator of bile acid homeostasis and hepatic lipogenesis. FXR has also been associated with Kupffer cells and liver sinusoidal endothelial cells of the liver, wherein it is believed to have functions related to inflammation, fibrosis, and portal hypertension.

A number of FXR agonists are known and are being investigated in connection with a number of physiological conditions, including liver diseases. FXR agonists can have benefits in steatosis, lobular inflammation, hepatocellular ballooning, and fibrosis.

FXR agonism can lead to different effects in different regions in the body. In the distal small intestine and systemically in organs such as the liver, activation of FXR directly causes the expression and secretion of the hormone FGF19. FGF19 modulates bile acid by down regulating bile acid synthesis, which can be beneficial for example in conditions such as liver disease. FXR agonists have also been associated with detrimental effects, such as pruritis. Such detrimental effects, and the degree to which they are experienced, could depend upon the site of FXR agonism. Pruritis, for instance, has been suggested to be associated with non-intestinal FXR agonism.

A need remains for FXR agonists with desirable potency, selectivity, and reduced detrimental effects.

### SUMMARY

The present disclosure provides compounds that bind to the NR1H4 receptor (FXR) and act as agonists or modulators of FXR. The disclosure further relates to the use of the compounds for the treatment and/or prophylaxis of diseases and/or conditions through binding of said nuclear receptor by said compounds.

The present disclosure provides a compound of Formula (I):

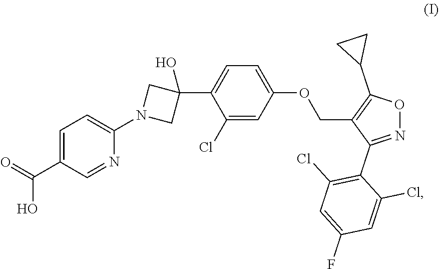 [SMILES: O=C(O)c1ccc(N2CC(O)(c3ccc(OCc4c(-c5c(Cl)cc(F)cc5Cl)noc4C4CC4)cc3Cl)C2)nc1]

or a pharmaceutically acceptable salt thereof.

Some embodiments provide for pharmaceutical compositions comprising a compound of Formula (I) and a pharmaceutically acceptable excipient.

Some embodiments provide solid forms of a compound of Formula (I) or a pharmaceutically acceptable salt thereof.

Also provided herein are methods of treating a patient having an FXR mediated condition comprising administering a compound of Formula (I) to a patient in need thereof.

### DESCRIPTION OF THE FIGURES

FIG. 1 depicts a chart of FGF19 plasma concentration over time in cynomolgus monkey after dosing with the compound of Example 1.

FIG. 2 depicts a chart of FGF19 plasma concentration over time in cynomolgus monkey after dosing with the compound of Comparative Example 1.

FIG. 3 depicts a chart of FGF19 plasma concentration over time in cynomolgus monkey after dosing with the compound of Comparative Example 2.

FIG. 4 is an X-ray powder diffractogram of Formula (I) mesylate Form I.

FIG. 5 is a DSC curve of Formula (I) mesylate Form I.

FIG. 6 is a TGA curve of Formula (I) mesylate Form I.

### DETAILED DESCRIPTION

The present disclosure relates to FXR agonists. The disclosure also relates to compositions and methods relating to FXR agonists and the use of such compounds for treatment and/or prophylaxis of diseases and conditions through binding of FXR by said compounds. The disclosure also relates to compositions and methods of treating and/or preventing liver disease including an FXR agonist in combination with one or more additional therapeutic agents.

FXR agonists are expressed in the liver and throughout the gastrointestinal tract, where their action or inaction can play a part in one or more diseases of the liver, such as NASH, PSC, and/or liver fibrosis. However, FXR agonists have also been identified in other regions of the body, in which case their function can vary. FGF19 is the primary target gene of FXR in the epithelial cells of the ileum. Physiological activation of ileal FXR by bile acids results in secretion of FGF19. In the liver, FXR agonism and FGF19 signaling have overlapping and distinct functions. For example, both pathways suppress bile acid synthesis. FXR agonism in hepatocytes indirectly downregulates many of the same enzymes that are reduced by FGF19.

FXR agonists can be useful in treating and preventing a variety of conditions, including liver disease. Liver diseases can include acute or chronic damages to the liver, for example, by infection, injury, abnormal build-up of normal substances in the blood, or other causes. Although many FXR agonists and related analogues are known, such FXR agonists can suffer from draw backs including poor efficacy, metabolism issues, and/or adverse events.

Disclosed herein are FXR agonists and related compositions and methods. FXR agonists disclosed herein can surprisingly maintain good therapeutic effect while minimizing adverse effects and adverse metabolism issues.

In some embodiments, FXR agonists described herein can have desirable cellular potency. For example, in some embodiments high cellular potency could provide higher FXR agonism with lower doses of administered drug relative to compounds having lower cellular potency.

In some embodiments, the present disclosure provides FXR agonists that can demonstrate high levels of FXR agonism in the gastrointestinal tract with reduced systemic FXR agonism. FXR agonists disclosed herein can cause increased FGF19 secretion upon oral dosing while resulting in minimal FGF19 increases upon intravenous dosing. Reduced systemic FXR agonism can be advantageous, for example by reducing and/or limiting the possibility of certain adverse reactions, such as pruritis, or by reducing risks of potential drug-drug interactions with systemic drugs.

In some embodiments FXR agonists described herein have good target selectivity. For example, FXR agonists described herein agonize FXR and do not significantly alter activity of TGR5 and/or other nuclear hormone receptors related to FXR. In some embodiments, FXR agonists described herein preferentially agonize intestinal FXR over hepatic FXR.

Advantageously, orally dosed FXR agonists disclosed herein can produce dose-dependent increases in plasma FGF19 levels and decreases in serum C4 levels, indicating reduced bile acid synthesis.

### Definitions and General Parameters

As used in the present specification, the following terms and phrases are generally intended to have the meanings as set forth below, except to the extent that the context in which they are used indicates otherwise.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

The disclosures illustratively described herein may suitably be practiced in the absence of any element or elements, limitation or limitations, not specifically disclosed herein. Thus, for example, the terms “comprising.” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

In some embodiments, the compounds of the present disclosure can be in the form of a “prodrug.” The term “prodrug” is defined in the pharmaceutical field as a biologically inactive derivative of a drug that upon administration to the human body is converted to the biologically active parent drug according to some chemical or enzymatic pathway. Examples of prodrugs include esterified carboxylic acids.

In the human liver, UDP-glucuronosyltransferases act on certain compounds having amino, carbamyl, thio (sulfhydryl) or hydroxyl groups to conjugate uridine diphosphate-α-D-glucuronic acid through glycoside bonds, or to esterify compounds with carboxy or hydroxyl groups in the process of phase II metabolism. Compounds of the present disclosure may be glucuronidated, that is to say, conjugated to glucuronic acid, to form glucuronides, particularly (β-D) glucuronides.

One step in the formation of bile is the conjugation of the individual bile acids with an amino acid, particularly glycine or taurine. Compounds of the present disclosure may be conjugated with glycine or taurine at a substitutable position.

The compounds of the present disclosure can be in the form of a pharmaceutically acceptable salt. The term “pharmaceutically acceptable salts” refers to salts prepared from pharmaceutically acceptable non-toxic bases or acids, including inorganic bases or acids and organic bases or acids. The compounds of the present disclosure can be in the form of a pharmaceutically acceptable salt. The term “pharmaceutically acceptable salts” refers to salts prepared from pharmaceutically acceptable non-toxic bases or acids, including inorganic bases or acids and organic bases or acids. In case the compounds of the present disclosure contain one or more acidic or basic groups, the disclosure also comprises their corresponding pharmaceutically or toxicologically acceptable salts, in particular their pharmaceutically utilizable salts. Thus, the compounds of the present disclosure which contain acidic groups can be present on these groups and can be used according to the disclosure, for example, as alkali metal salts, alkaline earth metal salts or ammonium salts. More precise examples of such salts include sodium salts, potassium salts, calcium salts, magnesium salts or salts with ammonia or organic amines such as, for example, ethylamine, ethanolamine, triethanolamine, amino acids, or other bases known to persons skilled in the art. The compounds of the present disclosure which contain one or more basic groups, i.e., groups which can be protonated, can be present and can be used according to the disclosure in the form of their addition salts with inorganic or organic acids. Examples of suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, and other acids known to persons skilled in the art.

If the compounds of the present disclosure simultaneously contain acidic and basic groups in the molecule, the disclosure also includes, in addition to the salt forms mentioned, inner salts or betaines (zwitterions). The respective salts can be obtained by customary methods which are known to the person skilled in the art like, for example, by contacting these with an organic or inorganic acid or base in a solvent or dispersant, or by anion exchange or cation exchange with other salts.

In some embodiments, a pharmaceutically acceptable salt of a compound of Formula (I) includes a zwitterion. For example, a compound of Formula (I) can form a zwitterion as follows:

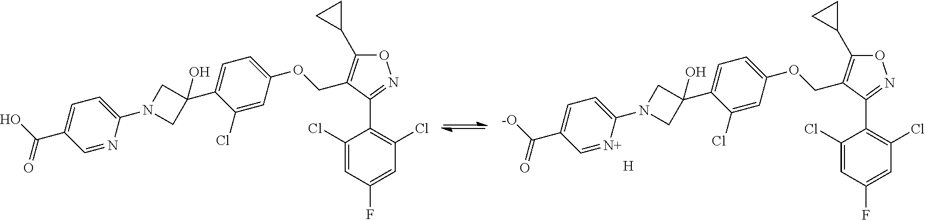

The present disclosure also includes all salts of the compounds of the present disclosure which, owing to low physiological compatibility, are not directly suitable for use in pharmaceuticals but which can be used, for example, as intermediates for chemical reactions or for the preparation of pharmaceutically acceptable salts. Acids and bases useful for reaction with an underlying compound to form pharmaceutically acceptable salts (acid addition or base addition salts respectively) are known to one of skill in the art. Similarly, methods of preparing pharmaceutically acceptable salts from an underlying compound (upon disclosure) are known to one of skill in the art and are disclosed in for example, Berge, et al. Journal of Pharmaceutical Science, January 1977 vol. 66, No. 1, and other sources.

Furthermore, compounds disclosed herein may be subject to tautomerism. Where tautomerism, e.g., keto-enol tautomerism, of compounds or their prodrugs may occur, the individual forms, like e.g., the keto and enol form, are each within the scope of the disclosure as well as their mixtures in any ratio. The same applies for stereoisomers, like e.g., enantiomers, cis/trans isomers, diastereomers, conformers and the like.

The term “protecting group” refers to a moiety of a compound that masks or alters the properties of a functional group or the properties of the compound as a whole. Chemical protecting groups and strategies for protection/deprotection are well known in the art. See. e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. The term “deprotecting” refers to removing the protecting group.

A “leaving group” includes a molecular fragment that can depart with a pair of electrons from a covalent bond to the reacting carbon atom during a chemical reaction.

It will be appreciated by the skilled person that when lists of alternative substituents include members which, because of their valency requirements or other reasons, cannot be used to substitute a particular group, the list is intended to be read with the knowledge of the skilled person to include only those members of the list which are suitable for substituting the particular group.

Further the compounds of the present disclosure may be present in the form of solvates, such as those which include as solvate water, or pharmaceutically acceptable solvates, such as alcohols, in particular ethanol. A “solvate” is formed by the interaction of a solvent and a compound.

In certain embodiments, provided are optical isomers, racemates, or other mixtures thereof of the compounds described herein or a pharmaceutically acceptable salt or a mixture thereof. If desired, isomers can be separated by methods well known in the art, e.g., by liquid chromatography. In those situations, the single enantiomer or diastereomer, i.e., optically active form, can be obtained by asymmetric synthesis or by resolution. Resolution can be accomplished, for example, by conventional methods such as crystallization in the presence of a resolving agent, or chromatography, using for example, a chiral high pressure liquid chromatography (HPLC) column.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposeable mirror images of one another. “Diastereomers” are stereoisomers that have at least two asymmetric atoms, but which are not mirror-images of each other.

Compounds disclosed herein and their pharmaceutically acceptable salts may, in some embodiments, include an asymmetric center and may thus give rise to enantiomers, diastereomers, and other stereoisomeric forms that may be defined, in terms of absolute stereochemistry, as (R)- or (S)- or, as (D)- or (L)- for amino acids. Some embodiments include all such possible isomers, as well as their racemic and optically pure forms. Optically active (+) and (−), (R)- and (S)-. or (D)- and (L)-isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using. for example, chiral high pressure liquid chromatography (HPLC). When the compounds described herein contain olefinic double bonds or other centres of geometric asymmetry, and unless specified otherwise, it is intended that the compounds include both E and Z geometric isomers.

Compositions provided herein that include a compound described herein or pharmaceutically acceptable salts, isomer, or a mixture thereof may include racemic mixtures, or mixtures containing an enantiomeric excess of one enantiomer or single diastereomers or diastereomeric mixtures. All such isomeric forms of these compounds are expressly included herein the same as if each and every isomeric form were specifically and individually listed.

Any formula or structure given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphoros, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Various isotopically labeled compounds of the present disclosure, for example those into which radioactive isotopes such as ³H, ¹³C, and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

The disclosure also includes “deuterated analogs” of compounds disclosed herein, in which from I to n hydrogens attached to a carbon atom is/are replaced by deuterium, in which n is the number of hydrogens in the molecule. Such compounds may exhibit increased resistance to metabolism and thus be useful for increasing the half-life of any compound of Formula (I) when administered to a mammal, e.g., a human. See, for example, Foster, “Deuterium Isotope Effects in Studies of Drug Metabolism,” Trends Pharmacol. Sci. 5 (12): 524-527 (1984). Such compounds are synthesized by means well known in the art, for example by employing starting materials in which one or more hydrogens have been replaced by deuterium.

Deuterium labelled or substituted therapeutic compounds of the disclosure may have beneficial DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life, reduced dosage requirements and/or an improvement in therapeutic index. An ¹⁸F labeled compound may be useful for PET or SPECT studies.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Accordingly, in the compounds of this disclosure any atom specifically designated as a deuterium (D) is meant to represent deuterium.

Furthermore, the present disclosure provides pharmaceutical compositions comprising a compound of the present disclosure, or a prodrug compound thereof, or a pharmaceutically acceptable salt or solvate thereof as active ingredient together with a pharmaceutically acceptable carrier.

“Pharmaceutical composition” means one or more active ingredients, and one or more inert ingredients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure can encompass any composition made by admixing at least one compound of the present disclosure and a pharmaceutically acceptable carrier.

As used herein, “pharmaceutically acceptable carrier” includes excipients or agents such as solvents, diluents, dispersion media, coatings, antibacterial and antifungal agents, isotonic and absorption delaying agents and the like that are not deleterious to the disclosed compound or use thereof. The use of such carriers and agents to prepare compositions of pharmaceutically active substances is well known in the art (see. e.g., Remington's Pharmaceutical Sciences, Mace Publishing Co., Philadelphia, PA 17th Ed. (1985); and Modern Pharmaceutics, Marcel Dekker, Inc. 3rd Ed. (G.S. Banker & C.T. Rhodes, Eds.)).

The terms “therapeutically effective amount” and “effective amount” are used interchangibly and refer to an amount of a compound that is sufficient to effect treatment as defined below, when administered to a patient (e.g., a human) in need of such treatment in one or more doses. The therapeutically effective amount will vary depending upon the patient, the disease being treated, the weight and/or age of the patient, the severity of the disease, or the manner of administration as determined by a qualified prescriber or care giver.

The term “treatment” or “treating” means administering a compound or pharmaceutically acceptable salt of formula (I) for the purpose of: (i) delaying the onset of a disease, that is, causing the clinical symptoms of the disease not to develop or delaying the development thereof: (ii) inhibiting the disease, that is, arresting the development of clinical symptoms: and/or (iii) relieving the disease, that is, causing the regression of clinical symptoms or the severity thereof.

List of Abbreviations and Acronyms

| Abbreviation | Meaning |
|---|---|
| (±)-BINAP | (±)-2,2′-Bis(diphenylphosphino)-1,1′-binaphthalene |
| 2-MeTHF | 2-Methyltetrahydrofuran |
| ACN or MeCN | Acetonitrile |
| aq. | Aqueous |
| Bn | Benzyl |
| BOC or Boc | t-Butyloxycarbonyl |
| BSA | Bovine serum albumin |
| BSS | Balanced Salt Solution |
| calcd | Calculated |
| DAST | (diethylamino)sulfur trifluoride |
| DCM | Dichloromethane |
| DIBAL-H | Diisobutylaluminum hydride |
| DMF | N,N-Dimethylformamide |
| DMSO | Dimethylsulfoxide |
| EA | Ethyl acetate |
| EDTA | Ethylenediaminetetraacetic acid |
| ESI | Electronspray Ionization |
| Et | Ethyl |
| Et₂O | Diethyl ether |
| EtOAc | Ethyl acetate |
| FBS | Fetal bovine serum |
| h or hr(s) | Hour(s) |
| HATU | 1-[Bis(dimethylamino)methylene]-1H-1,2,3- |
| | triazolo[4,5-b]pyridinium 3-oxid hexafluorophosphate |
| HPLC | High performance liquid chromatography |
| IPA | Isopropyl alcohol |
| IPTG | Isopropyl ß-D-1-thiogalactopyranoside |
| LCMS or LC/MS | Liquid Chromatography Mass Spectrometry |
| Me | Methyl |
| MEM | Minimum Essential Medium |
| MeOH | Methanol |
| MSA | Methanesulfonic acid |
| min | Minute(s) |
| MS | Mass Spectrometry |
| m/z | Mass-to-charge ratio |
| NADPH | Dihydronicotinamide-adenine dinucleotide phosphate |
| NMP | N-Methylpyrrolidone |
| NMR | Nuclear Magnetic Resonance spectroscopy |
| n-BuLi | n-Butyllithium |
| PE | Petroleum ether |
| rpm | Revolutions per minute |
| RT or rt | Room temperature |
| sat. | Saturated |
| TBAF | Tetrabutylammonium fluoride |
| TBDMS | tert-Butyldimethylsilyl |
| TBS | tert-Butyldimethylsilyl |
| TEMPO | 2,2,6,6-Tetramethylpiperidine 1-oxyl |
| TFA | Trifluoroacetic acid |
| THF | Tetrahydrofuran |
| TMS | Trimethylsilyl |
| UPLC | Ultra Performance Liquid Chromatography |

As used herein, a “FXR agonist” refers to any agent that is capable of binding and activating farnesoid X receptor (FXR) which may be referred to as bile acid receptor (BAR) or NR1H4 (nuclear receptor subfamily 1, group H, member 4) receptor. FXR agonist may act as agonists or partial agonists of FXR. The agent may be a chemical compound or biological molecule (e.g., a protein or antibody). The activity of a FXR agonist may be measured by several different methods, e.g., in an in vitro assay using the fluorescence resonance energy transfer (FRET) cell free assay as described in Pellicciari, et al. Journal of Medicinal Chemistry, 2002 vol. 15, No. 45:3569-72.

As referred to herein, an “ASK1 inhibitor” may be any agent that is capable of

inactivating an apoptosis signal regulating kinase 1 (ASK1) protein. The agent may be a chemical compound or biological molecule (e.g., a protein or antibody). The ASK1 protein activity may be measured by several different methods. For example, the activity of an ASK1 protein may be determined based on the ability of the ASK1 protein to phosphorylate a substrate protein. Methods for identifying an ASK1 inhibitor are known (see, e.g., U.S. 2007/0276050 and U.S. 2011/0009410, both of which are incorporated herein by reference in their entirety). Exemplary ASK1 substrate proteins include MAPKK3, MAPKK4, MAPKK6, MAPKK7, or fragments thereof. The ASK1 protein activity may also be measured by the phosphorylation level of the ASK1 protein, for example, the phosphorylation level of a threonine residue in the ASK1 protein corresponding to threonine 838 (T838) of a human full-length ASK1 protein or threonine 845 (T845) of a mouse full-length ASK1 protein. For example, where the ASK1 protein comprises a full-length human ASK1 protein sequence, an ASK1 inhibitor may attenuate phosphorylation of T838 in the full-length human ASK1 protein sequence. A site specific antibody against human ASK1 T838 or mouse ASK1 T845 may be used to detect the phosphohorylation level.

As used herein, an “ACC inhibitor” refers to any agent that is capable of binding and inhibiting Acetyl-CoA carboxylase (ACC). ACC inhibitors may act as inhibitors or partial inhibitors of ACC. The agent may be a chemical compound or biological molecule (e.g., a protein or antibody). The activity of an ACC inhibitor may be measured by methods known in the art, such as those described and cited in U.S. Pat. No. 8,969,557, and/or in U.S. Patent Application Publication No. 20160108061.

As used herein, a “Thyroid Hormone Receptor β agonist” or a “THR β agonist” refers to any agent that is capable of binding and activating thyroid hormone receptor beta, which may be referred to as NR1A2 (nuclear receptor subfamily 1, group A, member 2) receptor. THR β agonist may act as agonists or partial agonists of THR β. The agent may be a chemical compound or biological molecule (e.g., a protein or antibody). The activity of a THR β agonist may be measured by known methods.

### Compounds

Provided herein is a compound having the following Formula (I):

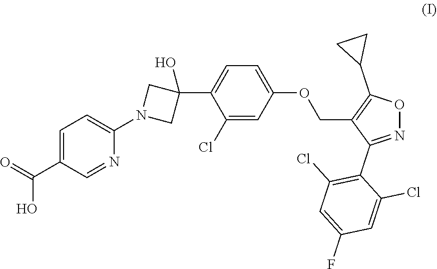 [SMILES: O=C(O)c1ccc(N2CC(O)(c3ccc(OCc4c(-c5c(Cl)cc(F)cc5Cl)noc4C4CC4)cc3Cl)C2)nc1]

or a pharmaceutically acceptable salt thereof.

In some embodiments, the pharmaceutically acceptable salt is a mesylate salt. For example, a compound of the following formula is provided:

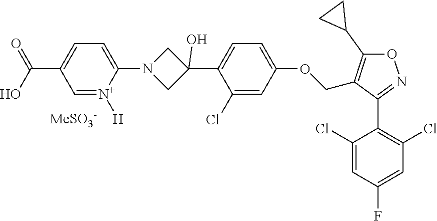 [SMILES: "MeSO3-" ; O=C(O)c1ccc(N2CC(O)(c3ccc(OCc4c(-c5c(Cl)cc(F)cc5Cl)noc4C4CC4)cc3Cl)C2)[nH+]c1]

### Solid Forms

In some embodiments, the present disclosure provides solid forms of a compound of Formula (I) or a pharmaceutically acceptable salt thereof. Solid forms, such as crystalline forms, can provide the advantage of bioavailability and stability that could be suitable for use as an active ingredient in a pharmaceutical composition.

Variations in the crystal structure of a pharmaceutical drug substance or active ingredient may affect the dissolution rate (which may affect bioavailability, etc.), manufacturability (e.g., ease of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control.

In some embodiments, a stable solid form of a compound of Formula (I) is provided. For example, a mesylate salt of Formula (I) can be produced as a stable crystalline form that does not convert to other polymorphic forms and/or that is formed as the same polymorphic form under a variety of manufacturing conditions.

Thus, crystalline forms of the compound of Formula (I) may provide advantages such as improving: the manufacturing process of the compound, the stability or storability of a drug product form of the compound, the stability or storability of a drug substance of the compound and/or the bioavailability and/or stability of the compound as an active agent.

In some embodiments, the solid form is Formula (I) mesylate Form I.

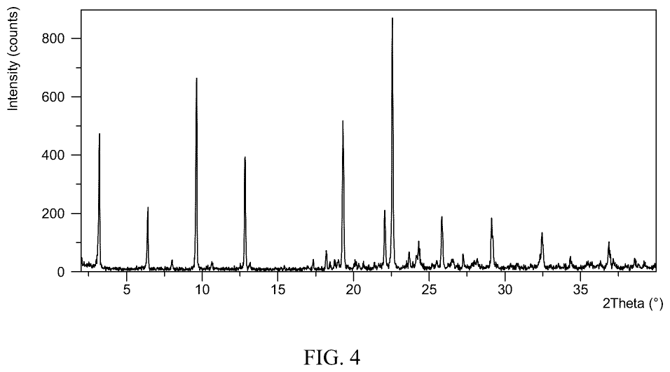

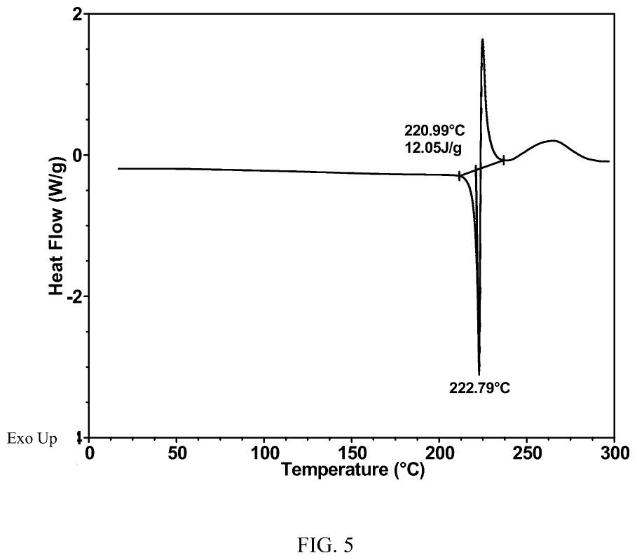

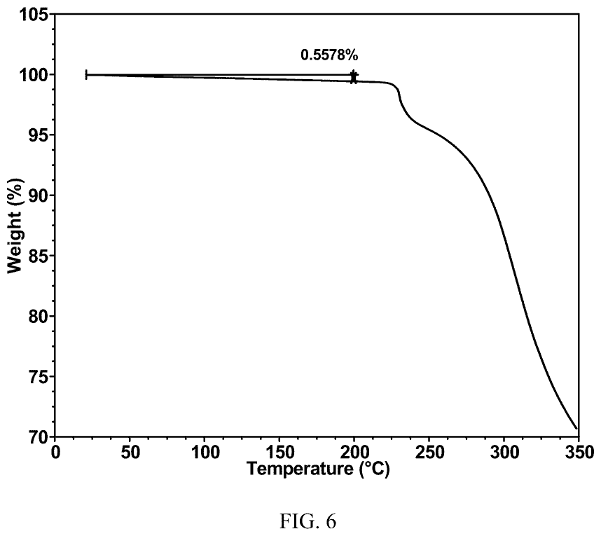

Formula (I) mesylate Form I can be characterized by an X-ray powder diffractogram having wherein the solid form exhibits an X-ray powder diffraction (XRPD) pattern substantially as shown in FIG. 4. Formula (I) mesylate Form I may exhibit a differential scanning calorimetry (DSC) thermogram substantially as shown in FIG. 5. Formula (I) mesylate Form I may exhibit a thermogravimetric analysis (TGA) thermogram substantially as shown in FIG. 6.

In some embodiments of Formula (I) mesylate Form I, at least one, at least two, or all of the following (a)-(c) apply: (a) Formula (I) mesylate Form I has an XRPD pattern substantially as shown in FIG. 4; (b) Formula (I) mesylate Form I has a DSC thermogram substantially as shown in FIG. 5; (c) Formula (I) mesylate Form I has a TGA thermogram substantially as shown in FIG. 6.

In some embodiments, Formula (I) mesylate Form I has at least one, at least two, or at least three of the following properties:
(a) an XRPD pattern substantially as shown in FIG. 4
(b) a DSC thermogram substantially as shown in FIG. 5
(c) a TGA thermogram substantially as shown in FIG. 6.

In some embodiments, Formula (I) mesylate Form I has an XRPD pattern displaying at least two, at least three, at least four, at least five, at least six, at least seven, at least eight, or at least nine of the degree 2θ-reflections with the greatest intensity as the XRPD pattern substantially as shown in FIG. 4.

In certain embodiments, Formula (I) mesylate Form I has an XRPD pattern comprising degree 2θ-reflections (±0.2 degrees 2θ) at 9.6, 19.3, and 22.6 degrees. In some embodiments, Formula (I) mesylate Form I has an XRPD pattern comprising degree 2θ-reflections (±0.2 degrees 2θ) at 9.6, 19.3, and 22.6 degrees and one, two or three of the degree 2θ-reflections (±0.2 degrees 2θ) at 3.2, 6.4, and 12.8 degrees. In some embodiments, Formula (I) mesylate Form I has an XRPD pattern comprising degree 2θ-reflections (±0.2 degrees 2θ) at 9.6, 19.3, and 22.6 degrees and one, two or three of the degree 2θ-reflections (±0.2 degrees 2θ) at 22.1, 25.8, and 29.1 degrees. In some embodiments, Formula (I) mesylate Form I has an XRPD pattern comprising degree 2θ-reflections (±0.2 degrees 2θ) at 9.6, 19.3, 22.6, 3.2, 6.4, 12.8, 22.1, 25.8, and 29.1 degrees.

In some embodiments, Formula (I) mesylate Form I has a differential scanning calorimetry thermogram comprising an endothermic peak with onset at about 221° C.

### Pharmaceutical Compositions and Modes of Administration

Furthermore, the present disclosure provides pharmaceutical compositions comprising at least one compound of the present disclosure, or a prodrug compound thereof, or a pharmaceutically acceptable salt or solvate thereof as active ingredient together with a pharmaceutically acceptable carrier.

The pharmaceutical composition of the present disclosure may additionally comprise one or more other compounds as active ingredients like a prodrug compound or other nuclear receptor modulators.

The compositions are suitable for oral, rectal, topical, parenteral (including subcutaneous, intramuscular, and intravenous), ocular (ophthalmic), pulmonary (nasal or buccal inhalation) or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of the present disclosure can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions: or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are employed. If desired, tablets may be coated by standard aqueous or non-aqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin: excipients such as dicalcium phosphate: a disintegrating agent such as corn starch, potato starch, alginic acid: a lubricant such as magnesium stearate: and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

In some embodiments, the compounds of the present disclosure may also be used as salts with various countercations to yield an orally available formulation.

The compounds of the present disclosure may also be administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Any suitable route of administration may be employed for providing a mammal, especially a human, with an effective dose of a compound of the present disclosure. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In some embodiments, compounds of the present disclosure are administered orally.

### Kits

Provided herein are also kits that include a compound of the disclosure, or a pharmaceutically acceptable salt, tautomer, stereoisomer, mixture of stereoisomers, prodrug, or deuterated analog thereof, and suitable packaging. In one embodiment, a kit further includes instructions for use. In one aspect, a kit includes a compound of the disclosure, or a pharmaceutically acceptable salt, tautomer, stereoisomer, mixture of stereoisomers, prodrug, or deuterated analog thereof, and a label and/or instructions for use of the compounds in the treatment of the indications, including the diseases or conditions, described herein.

Provided herein are also articles of manufacture that include a compound described herein or a pharmaceutically acceptable salt, tautomer, stereoisomer, mixture of stereoisomers, prodrug, or deuterated analog thereof in a suitable container. The container may be a vial, jar, ampoule, preloaded syringe, and intravenous bag.

### Treatment Methods and Uses

“Treatment” or “treating” is an approach for obtaining beneficial or desired results including clinical results. Beneficial or desired clinical results may include one or more of the following: (a) inhibiting the disease or condition (e.g., decreasing one or more symptoms resulting from the disease or condition, and/or diminishing the extent of the disease or condition): (b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or (c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in some embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy and/or veterinary applications. In some embodiments, the subject is a mammal. In one embodiment, the subject is a human.

The term “therapeutically effective amount” or “effective amount” of a compound described herein or a pharmaceutically acceptable salt, tautomer, stereoisomer, mixture of stereoisomers, prodrug, or deuterated analog thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition responsive to FXR agonism. The therapeutically effective amount may vary depending on the subject, and disease or condition being treated, the weight and age of the subject, the severity of the disease or condition, and the manner of administering, which can readily be determined by one or ordinary skill in the art.

The disclosure further relates to the use of compounds disclosed herein for the treatment and/or prophylaxis of diseases and/or conditions through binding of said nuclear receptor by said compounds. Further the present disclosure relates to the use of said compounds for the preparation of a medicament for the treatment and/or prophylaxis of diseases and/or conditions through binding of said nuclear receptor by said compounds.

In some embodiments, the present disclosure relates to the use of compounds according to Formula (I) in the preparation of a medicament for the prophylaxis and/or treatment of chronic intrahepatic or some forms of extrahepatic cholestatic conditions, of liver fibrosis, of acute intraheptic cholestatic conditions, of obstructive or chronic inflammatory disorders that arise out of improper bile composition, of gastrointestinal conditions with a reduced uptake of dietary fat and fat-soluble dietary vitamins, of inflammatory bowel diseases, of lipid and lipoprotein disorders, of Type II Diabetes and clinical complications of Type I and Type II Diabetes, of conditions and diseases which result from chronic fatty and fibrotic degeneration of organs due to enforced lipid and specifically triglyceride accumulation and subsequent activation of profibrotic pathways, of obesity and metabolic syndrome (combined conditions of dyslipidemia, diabetes and abnormally high body-mass index), of acute myocardial infarction, of acute stroke, of thrombosis which occurs as an endpoint of chronic obstructive atherosclerosis, of persistent infections by intracellular bacteria or parasitic protozoae, of non-malignant hyperproliferative disorders, of malignant hyperproliferative disorders, of colon adenocarcinoma and hepatocellular carcinoma in particular, of liver steatosis and associated syndromes, of liver failure or liver malfunction as an outcome of chronic liver diseases or of surgical liver resection, of Hepatitis B infection, of Hepatitis C infection and/or of cholestatic and fibrotic effects that are associated with alcohol-induced cirrhosis or with viral-borne forms of hepatitis.

Medicaments as referred to herein may be prepared by conventional processes, including the combination of a compound according to the present disclosure and a pharmaceutically acceptable carrier.

FXR can modulate both the synthetic output of bile acids in the liver and their recycling in the intestine (by regulating bile acid binding proteins). FXR can be involved in the regulation of many diverse physiological processes that are relevant in the etiology and for the treatment of diseases as diverse as cholesterol gallstones, metabolic disorders such as Type II Diabetes, dyslipidemias or obesity, chronic inflammatory diseases such as Inflammatory Bowel Diseases, or chronic intrahepatic forms of cholestasis.

FXR regulates a complex pattern of response genes in the liver and in the gastrointestinal tract. The gene products have impact on diverse physiological processes. For example. FXR represses the induction of Cyp7A1 via the upregulation of mRNA encoding SHP, a further nuclear receptor that is dominant repressive over LRH-1. Since FXR binds primary bile acids, the end products of this pathway, this can be regarded as an example of feedback inhibition on the gene expression level.

FXR ligands induce bile flow and change bile acid composition towards more hydrophilic composition. With the development of the first synthetic FXR ligand GW4064 as a tool compound and of the semi-synthetic artificial bile acid ligand 6-alpha-ethyl-CDCA, the effects of superstimulation of FXR by potent agonists could be analyzed. It was shown that both ligands induce bile flow in bile duct ligated animals. Moreover, in addition to choleretic effects, hepatoprotective effects could also be demonstrated. These hepatoprotective effects included anti-fibrotic effects resulting from the repression of Tissue Inhibitors of Matrix-Metalloproteinases TIMP-1 and 2, the induction of collagen-deposit resolving Matrix-Metalloproteinase 2 in hepatic stellate cells, and the subsequent reduction of alpha-collagen mRNA and Transforming growth factor beta (TGF-beta) mRNA, both of which are pro-fibrotic factors.

Furthermore, anti-cholestatic activity was demonstrated in bile-duct ligated animal models as well as in animal models of estrogen-induced cholestasis. Genetic studies demonstrate that in hereditary forms of cholestasis (Progressive Familiar Intrahepatic Cholestasis=PFIC, Type I-IV) either nuclear localization of FXR itself is reduced as a consequence of a mutation in the FICI gene (in PFIC Type I, also called Byler's Disease) (F. Chen et al., Gastroenterology 2004, 126, 756; L. Alvarez et al., Hum. Mol. Genet. 2004, 13, 2451) or levels of the FXR target gene encoding MDR-3 phospholipid export pump are reduced (in PFIC Type III). There is a growing body of evidence that FXR binding compounds can demonstrate substantial clinical utility in the therapeutic regimen of chronic cholestatic conditions such as Primary Biliary Cirrhosis (PBC) or Primary Sclerosing Cholangitis (PSC).

FXR agonists can be useful to prevent cholesterol gallstone formation or to prevent re-formation of gallstones after surgical removal or shockwave lithotripsy. For example, using the synthetic FXR tool compound GW4064 it could be demonstrated that activation of FXR leads to an improvement of the Cholesterol Saturation Index (CSI) and directly to an abolishment of gallstone formation in C57L gallstone susceptible mice whereas drug treatment in FXR knockout mice shows no effect on gallstone formation. Thus, in one embodiment of the disclosure, the compound according to Formula (I) and pharmaceutical compositions comprising said compound is used for the prophylaxis and/or treatment of obstructive or chronic inflammatory disorders that arise out of improper bile composition such as cholelithiasis also known as cholesterol gallstones.

FXR agonists can be useful in protecting the intestine from neoplastic transformation and from the development of polyps and their transition into adenocarcinoma in the gut. Absence of FXR leads to a high increase in the formation of Hepatocellular Cacrcinoma (HCC), the most prominent form of liver cancer. Whereas a functional FXR prevents the formation of colon adenocarcinoma and hepatocellular carcinoma, FXR activation induces liver regeneration after hepatectomy.

The combined hepatoprotective, anti-neoplastic and liver regenerative effects associated with FXR activation can be therapeutically exploited for the use of FXR agonists in the treatment of severe liver diseases. In one embodiment, the compounds according to the disclosure and pharmaceutical compositions comprising said compounds are used in the treatment of liver diseases such as HCC, stimulation of liver regrowth and amelioration of side effects associated with major liver resection, liver cirrhosis independent of the etiology and prevention or treatment of liver ischemia in the course of liver transplantation or major liver surgery.

Moreover, FXR can be a key regulator of serum triglycerides. Activation of FXR by synthetic agonists can leads to significant reduction of serum triglycerides, mainly in the form of reduced VLDL, but also to reduced total serum cholesterol. Lowering of serum triglycerides is not a stand alone effect. Treatment of db/db or ob/ob mice with synthetic FXR agonist GW4064 resulted in marked and combined reduction of serum triglycerides, total cholesterol, free fatty acids, ketone bodies such as 3-OH Butyrate. Moreover, FXR activation engages with the intracellular insulin signaling pathway in hepatocytes, resulting in reduced output of glucose from liver gluconeogenesis but concomitant increase in liver glycogen. Insulin sensitivity as well as glucose tolerance were positively impacted by FXR treatment. An effect on reduction of body weight was also recently observed in mice overfed with a high lipid diet. This weight loss effect might result from FXR's induction of FGF-19, a fibroblast growth factor that is known to lead to weight loss and athletic phenotype. The effect of FXR agonist on reduction of body weight has been demonstrated.

Accordingly, FXR agonists can be exploited in different therapeutic ways: FXR binding compounds are thought to be good candidates for the treatment of Type II Diabetes because of their insulin sensitization, glycogenogenic, and lipid lowering effects.

In one embodiment, the compounds according to the disclosure and pharmaceutical compositions comprising said compounds are used in the prophylaxis and/or treatment of Type II Diabetes which can be overcome by FXR-mediated upregulation of systemic insulin sensitivity and intracellular insulin signalling in liver, increased peripheral glucose uptake and metabolisation, increased glycogen storage in liver, decreased output of glucose into serum from liver-borne gluconeogenesis.

In a further embodiment, said compounds and pharmaceutical compositions are used for the prophylaxis and/or treatment of chronic intrahepatic, such as PBC, PSC, progressive familiar cholestasis (PFIC), alcohol-induced cirrhosis and associated cholestasis, and some forms of extrahepatic cholestatic conditions, or liver fibrosis.

The disclosure also relates to a compound of Formula (I) or a pharmaceutical composition comprising said compound for the prophylaxis and/or treatment of gastrointestinal conditions with a reduced uptake of dietary fat and fat-soluble dietary vitamins which can be overcome by increased intestinal levels of bile acids and phospholipids.

In a further embodiment, said compound or pharmaceutical composition is used for preventing and/or treating a disease selected from the group consisting of lipid and lipoprotein disorders such as hypercholesterolemia, hypertriglyceridemia, and atherosclerosis as a clinically manifest condition which can be ameliorated by FXR's beneficial effect on lowering total plasma cholesterol, lowering serum triglycerides, increasing conversion of liver cholesterol into bile acids and increased clearance and metabolic conversion of VLDL and other lipoproteins in the liver.

In one further embodiment, said compound and pharmaceutical composition are used for the prophylaxis and/or treatment of diseases where the combined lipid lowering, anti-cholestatic and anti-fibrotic effects of FXR-targeted medicaments can be exploited for the treatment of liver steatosis and associated syndromes such as Non-Alcoholic Steatohepatitis (NASH), or for the treatment of cholestatic and fibrotic effects that are associated with alcohol-induced cirrhosis, or with viral-borne forms of hepatitis.

In conjunction with the hypolipidemic effects it was shown that loss of functional FXR leads to increased atherosclerosis in ApoE knockout mice. Accordingly, FXR agonists can have clinical utility as anti-atherosclerotic and cardioprotective drugs. The downregulation of Endothelin-1 in Vascular Smooth Muscle Cells can also contribute to such beneficial therapeutic effects.

The disclosure also relates to a compound according to Formula (I) or a pharmaceutical composition comprising said compound for preventive and posttraumatic treatment of a cardiovascular disorder, such as acute myocardial infarction, acute stroke, or thrombosis which occur as an endpoint of chronic obstructive atherosclerosis.

Beyond controlling intestinal and colonic polyp formation, FXR seems to be expressed in breast cancer tissue and cell lines but not in healthy breast tissue and seems to interact with the Estrogen Receptor in ER positive breast cancer cells. Thus, FXR can be a potential target for the treatment of proliferative diseases, especially metastasizing cancer forms that express a small molecule responsive form of FXR.

In a further embodiment, said compounds and pharmaceutical compositions are used for the prophylaxis and/or treatment of malignant hyperproliferative disorders such as different forms of cancer, specifically certain forms of breast, liver or colon cancer where interference with an FXR ligand will have a beneficial impact.

FXR may be involved in the control of antibacterial defense in the intestine. FXR agonists can have a beneficial impact in the therapy of Inflammatory Bowel Disorders (IBD). For example, in IBD forms where the upper (ileal) part of the intestine is affected (e.g., ileal Crohn's disease) FXR agonists could have beneficial effects through FXR mediated control of bacterial growth. In IBD, the desensitization of the adaptive immune response is somehow impaired in the intestinal immune system. Bacterial overgrowth might then be the causative trigger towards establishment of a chronic inflammatory response. Hence, dampening of bacterial growth by FXR-borne mechanisms might be a key mechanism to prevent acute inflammatory episodes.

Thus, the disclosure also relates to a compound according to Formula (I) or a pharmaceutical composition comprising said compound for preventing and/or treating a disease related to an Inflammatory Bowel Disease, such as Crohn's disease or Colitis ulcerosa. FXR-mediated restoration of intestinal barrier function and reduction in non-commensal bacterial load is believed to be helpful in reducing the exposure of bacterial antigens to the intestinal immune system and can therefore reduce inflammatory responses.

The disclosure further relates to a compound or pharmaceutical composition for the prophylaxis and/or treatment of obesity and associated disorders such as metabolic syndrome (combined conditions of dyslipidemias, diabetes and abnormally high body-mass index) which can be overcome by FXR-mediated lowering of serum triglycerides, blood glucose and increased insulin sensitivity and FXR-mediated weight loss.

In a further embodiment, the compounds or pharmaceutical composition of the present disclosure are useful in preventing and/or treating clinical complications of Type I and Type II Diabetes. Examples of such complications include Diabetic Nephropathy, Diabetic Retinopathy, Diabetic Neuropathies, or Peripheral Arterial Occlusive Disease (PAOD). Other clinical complications of Diabetes are also encompassed by the present disclosure.

Furthermore, conditions and diseases which result from chronic fatty and fibrotic degeneration of organs due to enforced lipid and specifically triglyceride accumulation and subsequent activation of profibrotic pathways may also be prevented and/or treated by administering the compounds or pharmaceutical composition of the present disclosure. Such conditions and diseases encompass NASH and chronic cholestatic conditions in the liver, Glomerulosclerosis and Diabetic Nephropathy in the kidney, Macula Degeneration and Diabetic Retinopathy in the eye and neurodegenerative diseases, such as Alzheimer's Disease in the brain, or Diabetic Neuropathies in the peripheral nervous system.

In a further embodiment, the compounds or pharmaceutical composition of the present disclosure are useful in preventing and/or treating congenital hepatic fibrosis.

### Dosage

The effective dosage of active ingredient employed may vary depending on the particular compound employed, the mode of administration, the condition being treated and the severity of the condition being treated. Such dosage may be ascertained readily by a person skilled in the art.

When treating or preventing FXR mediated conditions for which compounds of the present disclosure are indicated, generally satisfactory results are obtained when the compounds of the present disclosure are administered at a daily dosage of from about 0.1 milligram to about 300 milligram per kilogram of animal body weight. In some embodiments, the compounds of the present disclosure are given as a single daily dose or in divided doses two to six times a day, or in sustained release form. For most large mammals, the total daily dosage is from about 1 milligram to about 1000 milligrams, or from about 1 milligram to about 50 milligrams. In the case of a 70 kg adult human, the total daily dose will generally be from about 7 milligrams to about 350 milligrams. This dosage regimen may be adjusted to provide the optimal therapeutic response. In some embodiments, the total daily dosage is from about 1 milligram to about 900 milligrams, about 10 milligrams to about 800 milligrams, about 20 milligrams to about 700 milligrams, about 30 milligrams to about 600 milligrams, about 40 milligrams to about 550 milligrams, about 50 milligrams to about 400 milligrams, about 50 milligrams to about 300 milligrams, or about 50 milligrams to about 200 milligrams.

The compounds of the present application or the compositions thereof may be administered once, twice, three, or four times daily, using any suitable mode described above. Also, administration or treatment with the compounds may be continued for a number of days: for example, commonly treatment would continue for at least 7 days, 14 days, or 28 days, for one cycle of treatment. Treatment cycles are well known in cancer chemotherapy, and are frequently alternated with resting periods of about 1 to 28 days, commonly about 7 days or about 14 days, between cycles. The treatment cycles, in other embodiments, may also be continuous.

In a particular embodiment, the methods provided herein comprise administering to the subject an initial daily dose of about 1 to 800 mg of a compound described herein and increasing the dose by increments until clinical efficacy is achieved. Increments of about 5, 10, 25, 50, or 100 mg can be used to increase the dose. The dosage can be increased daily, every other day, twice per week, or once per week.

### Combinations

In some embodiments, a compound disclosed herein is administered in combination with one or more additional therapeutic agents to treat or prevent a disease or condition disclosed herein. In some embodiments, the one or more additional therapeutic agents are a (n) ACE inhibitor, Acetyl COA carboxylase inhibitor, Adenosine A3 receptor agonist, Adiponectin receptor agonist, AKT protein kinase inhibitor, AMP-activated protein kinases (AMPK), Amylin receptor agonist, Angiotensin II AT-1 receptor antagonist, Autotaxin inhibitors, Bioactive lipid, Calcitonin agonist, Caspase inhibitor, Caspase-3 stimulator, Cathepsin inhibitor, Caveolin 1 inhibitor, CCR2 chemokine antagonist, CCR3 chemokine antagonist, CCR5 chemokine antagonist, Chloride channel stimulator, CNR1 inhibitor, Cyclin D1 inhibitor, Cytochrome P450 7A1 inhibitor, DGAT1/2 inhibitor, Dipeptidyl peptidase IV inhibitor, Endosialin modulator, Eotaxin ligand inhibitor, Extracellular matrix protein modulator, Farnesoid X receptor agonist, Fatty acid synthase inhibitors, FGF1 receptor agonist, Fibroblast growth factor (FGF-15, FGF-19, FGF-21) ligands, Galectin-3 inhibitor, Glucagon receptor agonist, Glucagon-like peptide 1 agonist, G-protein coupled bile acid receptor 1 agonist, Hedgehog (Hh) modulator, Hepatitis C virus NS3 protease inhibitor, Hepatocyte nuclear factor 4 alpha modulator (HNF4A), Hepatocyte growth factor modulator, HMG CoA reductase inhibitor, IL-10 agonist, IL-17 antagonist, Ileal sodium bile acid cotransporter inhibitor, Insulin sensitizer, integrin modulator, intereukin-1 receptor-associated kinase 4 (IRAK4) inhibitor, Jak2 tyrosine kinase inhibitor, ketohexokinase inhibitors, Klotho beta stimulator, 5-Lipoxygenase inhibitor, Lipoprotein lipase inhibitor, Liver X receptor, LPL gene stimulator, Lysophosphatidate-1 receptor antagonist, Lysyl oxidase homolog 2 inhibitor, Matrix metalloproteinases (MMPs) inhibitor, MEKK-5 protein kinase inhibitor, Membrane copper amine oxidase (VAP-1) inhibitor, Methionine aminopeptidase-2 inhibitor, Methyl CpG binding protein 2 modulator, MicroRNA-21 (miR-21) inhibitor, Mitochondrial uncoupler, Myelin basic protein stimulator, NACHT LRR PYD domain protein 3 (NLRP3) inhibitor, NAD-dependent deacetylase sirtuin stimulator, NADPH oxidase inhibitor (NOX), Nicotinic acid receptor 1 agonist, P2Y13 purinoceptor stimulator, PDE 3 inhibitor, PDE 4 inhibitor, PDE 5 inhibitor, PDGF receptor beta modulator, Phospholipase C inhibitor, PPAR alpha agonist, PPAR delta agonist, PPAR gamma agonist, PPAR gamma modulator, Protease-activated receptor-2 antagonist, Protein kinase modulator, Rho associated protein kinase inhibitor, Sodium glucose transporter-2 inhibitor, SREBP transcription factor inhibitor, STAT-1 inhibitor, Stearoyl CoA desaturase-1 inhibitor, Suppressor of cytokine signalling-1 stimulator, Suppressor of cytokine signalling-3 stimulator, Transforming growth factor β (TGF-β), Transforming growth factor β activated Kinase 1 (TAK1), Thyroid hormone receptor beta agonist, TLR-4 antagonist, Transglutaminase inhibitor, Tyrosine kinase receptor modulator, GPCR modulator, nuclear hormone receptor modulator, WNT modulators, or YAP/TAZ modulator.

Non-limiting examples of the one or more additional therapeutic agents include:
ACE inhibitors, such as enalapril;
Acetyl CoA carboxylase (ACC) inhibitors, such as DRM-01, gemcabene, PF-05175157, and QLT-091382;
Adenosine receptor agonists, such as CF-102, CF-101, CF-502, and CGS21680;
Adiponectin receptor agonists, such as ADP-355;
Amylin/calcitonin receptor agonists, such as KBP-042;
AMP activated protein kinase stimulators, such as O-304;
Angiotensin II AT-1 receptor antagonists, such as irbesartan;
Autotaxin inhibitors, such as PAT-505, PAT-048, GLPG-1690, X-165, PF-8380, and AM-063;
Bioactive lipids, such as DS-102;
Cannabinoid receptor type 1 (CNR1) inhibitors, such as namacizumab and GWP-42004;
Caspase inhibitors, such as emricasan;
Pan cathepsin B inhibitors, such as VBY-376;
Pan cathepsin inhibitors, such as VBY-825;
CCR2/CCR5 chemokine antagonists, such as cenicriviroc;
CCR2 chemokine antagonists, such as propagermanium;
CCR3 chemokine antagonists, such as bertilimumab;
Chloride channel stimulators, such as cobiprostone;
Diglyceride acyltransferase 2 (DGAT2) inhibitors, such as IONIS-DGAT2Rx;
Dipeptidyl peptidase IV inhibitors, such as linagliptin;
Eotaxin ligand inhibitors, such as bertilimumab;
Extracellular matrix protein modulators, such as CNX-024;
Fatty acid synthase inhibitors, such as TVB-2640;
Fibroblast growth factor 19 (rhFGF19)/cytochrome P450 (CYP) 7A1 inhibitors, such as NGM-282;
Fibroblast growth factor 21 (FGF-21) ligand, such as BMS-986171, BMS-986036;
Fibroblast growth factor 21 (FGF-21)/glucagon like peptide 1 (GLP-1) agonists, such as YH-25723;
Galectin-3 inhibitors, such as GR-MD-02;
Glucagon-like peptide 1 (GLP1R) agonists, such as AC-3174, liraglutide, semaglutide;
G-protein coupled bile acid receptor 1 (TGR5) agonists, such as RDX-009, INT-777;
Heat shock protein 47 (HSP47) inhibitors. such as ND-L02-s0201;
HMG CoA reductase inhibitors, such as atorvastatin, fluvastatin, pitavastatin, pravastatin, rosuvastatin, and simvastatin;
IL-10 agonists, such as peg-ilodecakin;
Ileal sodium bile acid cotransporter inhibitors, such as A-4250, volixibat potassium ethanolate hydrate (SHP-262), and GSK2330672;
Insulin sensitizers, such as, KBP-042, MSDC-0602K, Px-102, RG-125 (AZD4076), and VVP-100X;
Ketohexokinase inhibitors, such as PF-06835919;
beta Klotho (KLB)-FGF1c agonist, such as NGM-313;
5-Lipoxygenase inhibitors, such as tipelukast (MN-001);
Lipoprotein lipase inhibitors, such as CAT-2003;
LPL gene stimulators, such as alipogene tiparvovec;
Liver X receptor (LXR) modulators, such as PX-L603, PX-L493, BMS-852927, T-0901317, GW-3965, and SR-9238;
Lysophosphatidate-1 receptor antagonists, such as BMT-053011, UD-009, AR-479, ITMN-10534, BMS-986020, and KI-16198;
Lysyl oxidase homolog 2 inhibitors, such as simtuzumab;
Semicarbazide-Sensitive Amine Oxidase/Vascular Adhesion Protein-1 (SSAO/VAP-1) Inhibitors, such as PXS-4728A;
Methionine aminopeptidase-2 inhibitors, such as ZGN-839;
Methyl CpG binding protein 2 modulators, such as mercaptamine;
Mitochondrial uncouplers, such as 2,4-dinitrophenol;
Myelin basic protein stimulators, such as olesoxime;
NADPH oxidase 1/4 inhibitors, such as GKT-831;
Nicotinic acid receptor 1 agonists, such as ARI-3037MO;
Nitazoxinide;
NACHT LRR PYD domain protein 3 (NLRP3) inhibitors, such as KDDF-201406-03, and NBC-6;
Nuclear receptor modulators, such as DUR-928;
P2Y13 purinoceptor stimulators, such as CER-209;
PDE 3/4 inhibitors, such as tipelukast (MN-001);
PDE 5 inhibitors, such as sildenafil:
PDGF receptor beta modulators, such as BOT-191, BOT-509;
PPAR agonists, such as elafibranor (GFT-505), MBX-8025, deuterated pioglitazone R-enantiomer, pioglitazone, DRX-065, saroglitazar, and IVA-337;
Protease-activated receptor-2 antagonists, such as PZ-235;
Protein kinase modulators, such as CNX-014;
Rho associated protein kinase (ROCK) inhibitors, such as KD-025;
Sodium glucose transporter-2 (SGLT2) inhibitors, such as ipragliflozin, remogliflozin etabonate, ertugliflozin, dapagliflozin, and sotagliflozin;
SREBP transcription factor inhibitors, such as CAT-2003 and MDV-4463;
Stearoyl CoA desaturase-1 inhibitors, such as aramchol;
Thyroid hormone receptor (THR) beta agonists, such as MGL-3196, MGL-3745, VK-2809;
TLR-4 antagonists, such as JKB-121;
Tyrosine kinase receptor modulators, such as CNX-025;
GPCR modulators, such as CNX-023; and
Nuclear hormone receptor modulators, such as Px-102.

In certain specific embodiments, the one or more additional therapeutic agents are selected from A-4250, AC-3174, acetylsalicylic acid, AK-20, AKN-083, alipogene tiparvovec, aramchol, ARI-3037MO, ASP-8232, atorvastatin, bertilimumab, Betaine anhydrous, BAR-704, BI-1467335, BMS-986036, BMS-986171, BMT-053011, BOT-191, BTT-1023, BWD-100, BWL-200, CAT-2003, cenicriviroc, CER-209, CF-102, CGS21680, CNX-014, CNX-023, CNX-024, CNX-025, cobiprostone, colesevelam, dapagliflozin, 16-dehydro-pregnenolone, deuterated pioglitazone R-enantiomer, 2,4-dinitrophenol, DRX-065, DS-102, DUR-928, EDP-305, elafibranor (GFT-505), emricasan, enalapril, EP-024297, ertugliflozin, evogliptin, EYP-001, F-351, fexaramine, GKT-831, GNF-5120, GR-MD-02, hydrochlorothiazide, icosapent ethyl ester, IMM-124-E, INT-767, IONIS-DGAT2Rx, INV-33, ipragliflozin, Irbesarta, propagermanium, IVA-337, JKB-121, KB-GE-001, KBP-042, KD-025, M790, M780, M450, metformin, sildenafil, LC-280126, linagliptin, liraglutide, LJN-452, LMB-763, MBX-8025, MDV-4463, mercaptamine, MET-409, MGL-3196, MGL-3745, MSDC-0602K, namacizumab, NC-101, ND-L02-s0201, NFX-21, NGM-282, NGM-313, NGM-386, NGM-395, NTX-023-1, norursodeoxycholic acid, 0-304, obeticholic acid, 25HC3S, olesoxime, PAT-505, PAT-048, peg-ilodecakin, pioglitazone, pirfenidone, PRI-724, PX20606, Px-102, PX-L603, PX-L493, PXS-4728A, PZ-235, RDX-009, RDX-023, remogliflozin etabonate, repurposed tricaprilin, RG-125 (AZD4076), saroglitazar, semaglutide, simtuzumab, SIPI-7623, solithromycin, sotagliflozin, statins (atorvastatin, fluvastatin, pitavastatin, pravastatin, rosuvastatin, simvastatin), TCM-606F, TERN-101, TEV-45478, tipelukast (MN-001), TLY-012, tropifexor, TRX-318, TVB-2640, UD-009, ursodeoxycholic acid, VBY-376, VBY-825, VK-2809, vismodegib, volixibat potassium ethanolate hydrate (SHP-626), VVP-100X, WAV-301, WNT-974, and ZGN-839.

In some embodiments, methods and compositions include a therapeutically effective amount of an Apoptosis Signal-Regulating Kinase 1 (ASK1) inhibitor and a therapeutically effective amount of a Farnesoid X Receptor (FXR) agonist, wherein the FXR agonist is a compound of Formula (I):

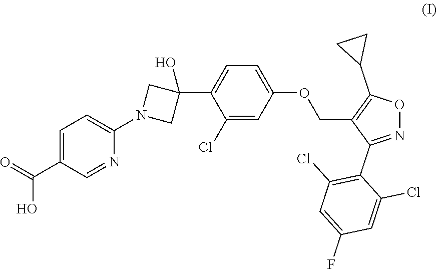 [SMILES: O=C(O)c1ccc(N2CC(O)(c3ccc(OCc4c(-c5c(Cl)cc(F)cc5Cl)noc4C4CC4)cc3Cl)C2)nc1]

or a pharmaceutically acceptable salt, a stereoisomer, a mixture of stereoisomers, or a tautomer thereof.

In certain embodiments of the methods and pharmaceutical compositions disclosed herein, the ASK1 inhibitor is a compound of Formula (II):

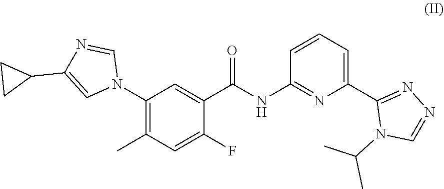 [SMILES: Cc1cc(F)c(C(=O)Nc2cccc(-c3nncn3C(C)C)n2)cc1-n1cnc(C2CC2)c1]

or a pharmaceutically acceptable salt, a stereoisomer, a mixture of stereoisomers, or a tautomer thereof.

ASK1 inhibitors, such as the compound of Formula (II), can be synthesized and characterized using methods known to those of skill in the art, such as those described in U.S. 2007/0276050 U.S. 2011/0009410, and U.S. 2013/0197037.

In some embodiments, methods and compositions include a therapeutically effective amount of an Acetyl COA Carboxylase (ACC) inhibitor and a therapeutically effective amount of a Farnesoid X Receptor (FXR) agonist, wherein the FXR agonist is a compound of Formula (I):

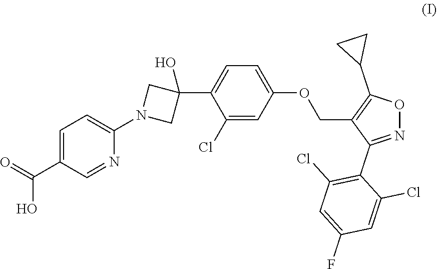 [SMILES: O=C(O)c1ccc(N2CC(O)(c3ccc(OCc4c(-c5c(Cl)cc(F)cc5Cl)noc4C4CC4)cc3Cl)C2)nc1]

or a pharmaceutically acceptable salt, a stereoisomer, a mixture of stereoisomers, or a tautomer thereof.

In certain embodiments of the methods and pharmaceutical compositions disclosed herein, the ACC inhibitor is a compound of Formula (III):

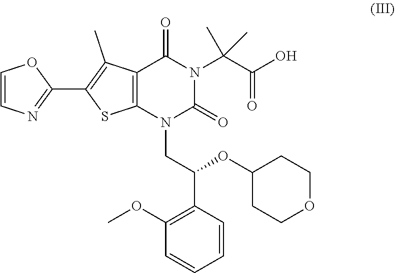 [SMILES: COc1ccccc1[C@H](Cn1c(=O)n(C(C)(C)C(=O)O)c(=O)c2c(C)c(-c3ncco3)sc21)OC1CCOCC1]

or a pharmaceutically acceptable salt thereof.

ACC inhibitors, such as the compound of Formula (III), can be synthesized and characterized using methods known to those of skill in the art, such as those described in PCT International Application Publication No. WO 2013/071169.

In some embodiments, methods and compositions include a therapeutically effective amount of a Thyroid Hormone Receptor (THR) β agonist in combination with a therapeutically effective amount of a Farnesoid X Receptor (FXR) agonist, wherein the FXR agonist is a compound of Formula (I):

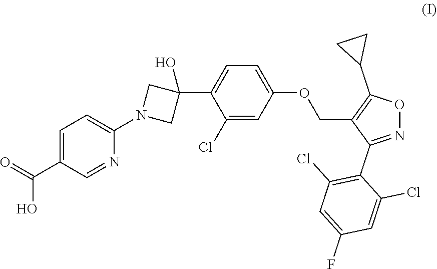 [SMILES: O=C(O)c1ccc(N2CC(O)(c3ccc(OCc4c(-c5c(Cl)cc(F)cc5Cl)noc4C4CC4)cc3Cl)C2)nc1]

or a pharmaceutically acceptable salt, a stereoisomer, a mixture of stereoisomers, or a tautomer thereof.

In certain embodiments of the methods and pharmaceutical compositions disclosed herein, the THR β agonist is a compound of Formula (IV):

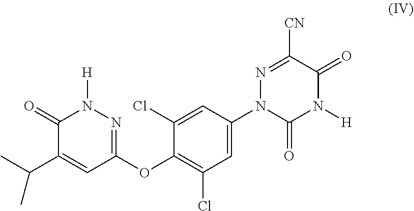 [SMILES: CC(C)c1cc(Oc2c(Cl)cc(-n3nc(C#N)c(=O)[nH]c3=O)cc2Cl)n[nH]c1=O]

or a pharmaceutically acceptable salt, a stereoisomer, a mixture of stereoisomers, or a tautomer thereof.

THR β agonists, such as the compound of Formula (IV), can be synthesized and characterized using methods known to those of skill in the art.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that these examples are exemplary and not exhaustive. Many changes can be made in the specific embodiments which are disclosed and still obtain a like or similar result without departing from the spirit and scope of the disclosure.

Compounds disclosed herein can be prepared according to the procedures of the following Schemes and Examples, using appropriate materials and are further exemplified by the following specific examples. Moreover, by utilizing the procedures described herein, in conjunction with ordinary skills in the art, additional compounds of the present disclosure claimed herein can be readily prepared. The examples further illustrate details for the preparation of the compounds of the present disclosure. Those skilled in the art will readily understand that known variations of the conditions and processes of the following preparative procedures can be used to prepare these compounds. For synthesizing compounds which are embodiments described in the present disclosure, inspection of the structure of the compound to be synthesized will provide the identity of each substituent group. In some cases, the identity of the final product can render apparent the identity of the necessary starting materials by a process of inspection, given the examples herein. Compounds can be isolated in the form of their pharmaceutically acceptable salts, such as those described above. Compounds described herein are typically stable and isolatable at room temperature and pressure.

An illustration of the preparation of compounds disclosed herein is shown below. Unless otherwise indicated, variables have the same meaning as described above. The examples presented below are intended to illustrate particular embodiments of the disclosure. Suitable starting materials, building blocks and reagents employed in the synthesis as described below are commercially available from Sigma-Aldrich or Acros Organics, for example, or can be routinely prepared by procedures described in the literature, for example in “March's Advanced Organic Chemistry: Reactions, Mechanisms, and Structure”, 5^{th} Edition; John Wiley & Sons or T. Eicher, S. Hauptmann “The Chemistry of Heterocycles: Structures, Reactions, Synthesis and Application”, 2^{nd} edition, Wiley-VCH 2003: Fieser et al. “Fiesers' Reagents for Organic Synthesis” John Wiley & Sons 2000.

### Methods

### X-Ray Powder Diffraction (XRPD)

XRPD patterns were collected on a PANanalytical XPERT-PRO diffractometer at ambient conditions under the following experimental settings: 45 KV, 40 mA, Kα1=1.5406 Å, scan range 2 to 40° 2θ, step size 0.0084 or 0.0167°2θ, measurement time: 5 min.

### Differential Scanning Calorimetry (DSC)

DSC thermograms were collected on a TA Instruments Q2000 system equipped with a 50 position auto-sampler. The calibration for energy and temperature was carried out using certified indium. Typically 1-5 mg of each sample, in a pin-holed aluminium pan, was heated at 10° C./min from about 25° C. to about 300° C. A purge of dry nitrogen at 50 mL/min was maintained over the sample throughout the measurement. The onset of the melting endotherm was reported as the melting point.

### Thermogravimetric Analysis (TGA)

TGA thermograms were collected on a TA Instruments Q5000 system, equipped with a 25 position auto-sampler. About 1-5 mg of each sample was loaded onto a pre-tared aluminium pan and heated at 10° C./min from about 25° C. to about 350° C. A nitrogen purge at 25 mL/min was maintained over the sample throughout the measurement.

### General Synthetic Scheme

Compounds of Formula (Ia) wherein Y is N can be synthesized according to the following general synthetic schemes.

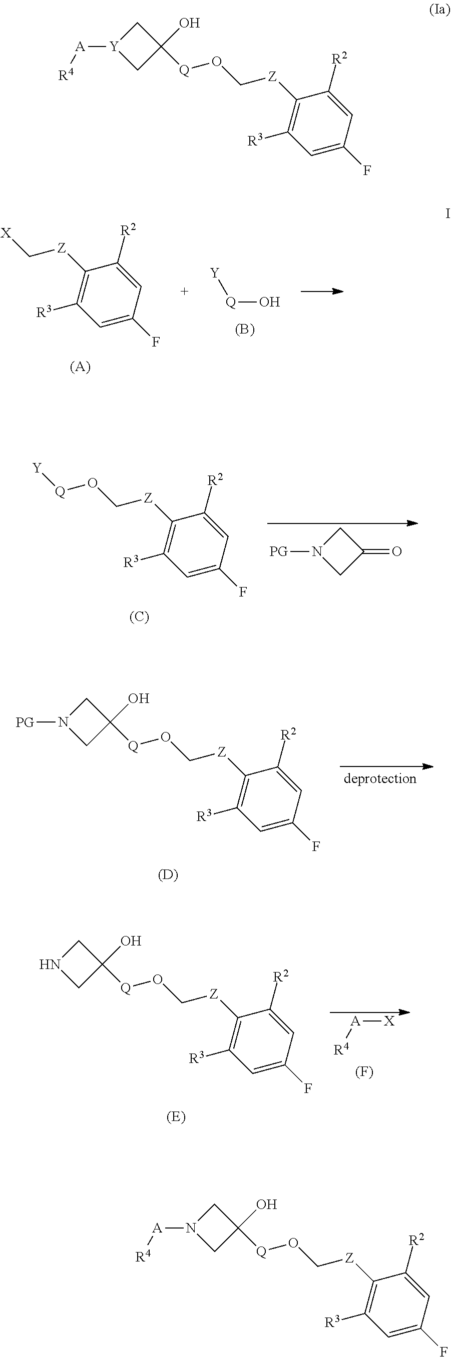

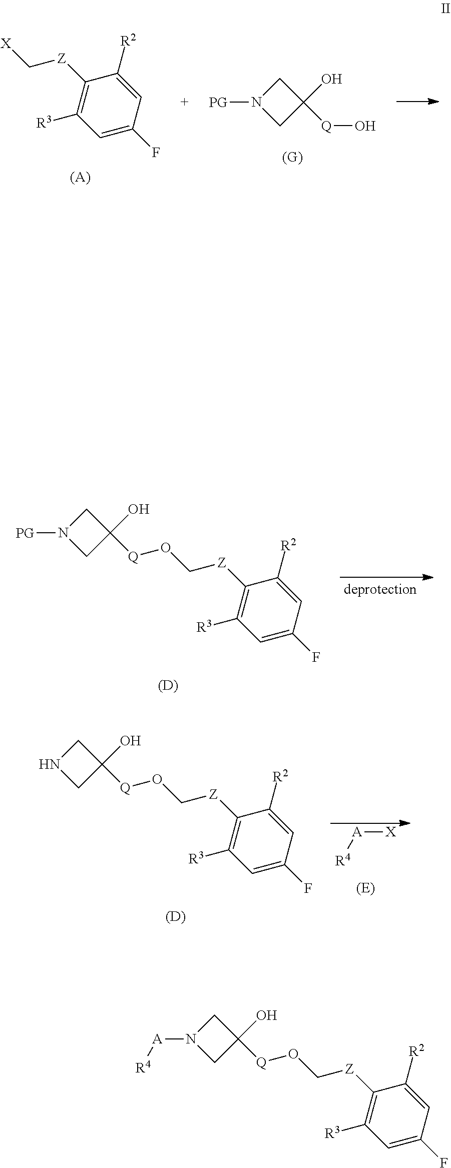

In the general synthetic schemes above, A is pyridylene or phenylene, each of which is optionally substituted with one or two groups independently selected from halogen, C₁₋₄-alkoxy, halo-C₁₋₄-alkoxy, C₁₋₄-alkyl, and halo-C₁₋₄-alkyl: Q is phenylene or pyridylene, each of which is optionally substituted with one or two substituents independently selected from halogen, methyl, C₁₋₄-alkoxy, halo-C₁₋₄-alkoxy, —CH₂F, —CHF₂, and —CF₃; X is a leaving group, Y is a group such as a halogen that can be used for the formation of an organometallic species such as a Grignard reagent, Z is isoxazole substituted with Ror pyrazole substituted with C₁₋₄-alkyl or C₃₋₆-cycloalkyl, R² and R³ are independently selected from hydrogen, halogen, methoxy, —CF₃, —CHF₂, —CH₂F, —OCH₂F, —OCHF₂, —OCF₃, and methyl; R⁴ is —CO₂R⁵ or —C(O)NR⁵R⁶; R⁵ is hydrogen, C₁₋₆-alkyl, or halo-C₁₋₆-alkyl: and R⁶ is hydrogen or C₁₋₆-alkyl, wherein said C₁₋₆-alkyl is optionally substituted with 1 to 6 substituents independently selected from halogen, —SO₃H, and —CO₂H. PG is a protecting group, and the remaining variables are as provided herein. A compound of Formula (C) can be prepared by reacting a compound of Formula (A) with a compound of Formula (B) in the presence of a base. A compound of Formula (D) is formed from a compound of Formula (C) through the formation of an organometallic species such as a Grignard reagent followed by condensation with an appropriately-protected 3-ketoazetidine. A compound of Formula (E) is formed from a compound of Formula (D) under appropriate deprotection conditions. A compound of Formula (E) can be combined with a compound of Formula (F) in the presence of a base to give a compound of Formula (Ia).

Alternatively, a compound of Formula (D) can be formed by reacting a compound of Formula (A) with a compound of Formula (G) in the presence of a base.

Appropriate compounds of structure (A) and (B) can be prepared according to the methods described in the following Examples or by methods known in the art. For example, X can be a halogen (e.g., fluoro, bromo, chloro, and/or iodo) and PG can be tert-butylcarbonyl protecting group (BOC).

### Example 1: Preparation of Formula (I)

Step 1: Preparation of 2,6-dichloro-4-fluorobenzaldehyde oxime

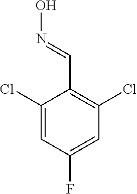 [SMILES: O/N=C/c1c(Cl)cc(F)cc1Cl]

A suspension of 2,6-dichloro-4-fluorobenzaldehyde (20 g, 100 mmol), hydroxylamine hydrochloride (14 g, 210 mmol), and sodium carbonate (27 g, 260 mmol) in ethanol/water (5:1, 170 mL) was sonicated for about 10 minutes and then left to stir overnight at room temperature.

The mixture was diluted with water and ethyl acetate. The aqueous phase was extracted twice with ethyl acetate. The combined organic layers were washed with saturated aqueous sodium chloride solution, dried over anhydrous magnesium sulfate, filtered, and concentrated under reduced pressure to provide the desired intermediate. LCMS-ESI⁺ (m/z): [M+H]⁺calcd for C₇H₅Cl₂FNO: 207.97; found: 207.99.

Step 2: Preparation of 2,6-dichloro-4-fluoro-N-hydroxybenzimidoyl chloride

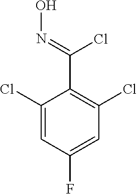 [SMILES: O/N=C(\Cl)c1c(Cl)cc(F)cc1Cl]

A solution of 2,6-dichloro-4-fluorobenzaldehyde oxime (21 g, 99 mmol) in N,N-dimethylformamide (200 mL) was treated with a single portion of N-chlorosuccinimide (1.5 g, 11 mmol). Hydrogen chloride vapor (approximately 40 mL, taken from headspace of concentrated hydrochloric acid bottle) was bubbled into the mixture. Following the addition of another portion of N-chlorosuccinimide (1.5 g, 90 mmol), two more volumes of hydrogen chloride gas (40 mL×2) were introduced. Additional N-chlorosuccinimide (12 g, 11 mmol) was added in small portions. At the observation of an exotherm to 25° C., the mixture was cooled in room temperature water bath. The temperature was allowed to fall to about 23° C. and the remainder of the N-chlorosuccinimide was added portionwise. At the end of the addition, LC/MS analysis revealed the persistence of the oxime starting material, so a final portion of N-chlorosuccinimide (1.2 g, 9.0 mmol) was added. The mixture was allowed to stir overnight at room temperature and was carried forward without work up to the subsequent synthetic step. LCMS-ESI⁺ (m/z): [M+H]⁺ calcd for C₇H₄Cl₃FNO: 241.93; found: 242.20.

Step 3: Preparation of ethyl 5-cyclopropyl-3-(2,6-dichloro-4-fluorophenyl)isoxazole-4carboxylate

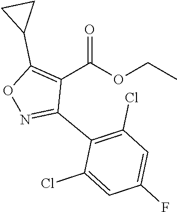 [SMILES: CCOC(=O)c1c(-c2c(Cl)cc(F)cc2Cl)noc1C1CC1]

A solution of ethyl cyclopropyl-3-oxopropanoate (19 g, 120 mmol) in 2-methyltetrahydrofuran at room temperature was treated via syringe with triethylamine (55 mL, 400 mmol). After 30 minutes of stirring, the reaction mixture containing 2,6-dichloro-4-fluoro-N-hydroxybenzimidoyl chloride (24 g, 99 mmol) was added dropwise via syringe. At the end of the addition, the mixture was heated at about 60° C. overnight. The reaction mixture was concentrated under reduced pressure. The residue was diluted with 10% hydrochloric acid solution and ethyl acetate. The aqueous phase was extracted three times with ethyl acetate. The combined organic extracts were washed once with saturated aqueous sodium chloride solution, dried over anhydrous magnesium sulfate, and concentrated under reduced pressure. The residue was purified by flash chromatography (silica gel) to provide the desired intermediate. LCMS-ESI⁺ (m/z): [M+H]⁺ calcd for C₁₅H₁₃Cl₂FNO₃: 344.02: found: 344.03.

Step 4: Preparation of (5-cyclopropyl-3-(2,6-dichloro-4-fluorophenyl)isoxazol-4-yl)methanol

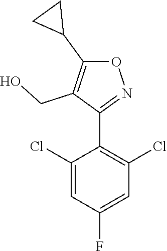 [SMILES: OCc1c(-c2c(Cl)cc(F)cc2Cl)noc1C1CC1]

A solution of (ethyl 5-cyclopropyl-3-(2,6-dichloro-4-fluorophenyl)isoxazole-4-carboxylate (15 g, 44 mmol) was taken up in 2-methyltetrahydrofuran (220 mL) and was cooled with magnetic stirring in an about −12 to −10° C. wet ice/acetone bath. Lithium aluminum hydride solution (2.0 M in tetrahydrofuran, 53 mmol) was added dropwise. After 30 minutes of stirring in the bath, the mixture, cooled in an ice-water bath, was quenched successively with water (2.0 mL, dropwise very carefully), 15% aqueous sodium hydroxide solution (2.0 mL), and water (6.0 mL). The suspension was allowed to stir at room temperature for 15 minutes before the addition of anhydrous magnesium sulfate, which was followed by another hour of stirring. The slurry was filtered: the filter cake was washed with ethyl acetate, and filtrate was concentrated to dryness under reduced pressure. The residue was purified by flash chromatography (silica gel) to provide the desired intermediate. LCMS-ESI⁺ (m/z): [M+H]⁺ calcd for C₁₃H₁₁Cl₂FNO₂: 302.01: found: 302.51.

Step 5: Preparation of 4-(chloromethyl)-5-cyclopropyl-3-(2,6-dichloro-4-fluorophenyl)isoxazole

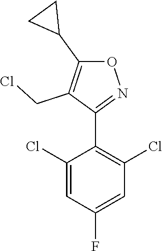 [SMILES: Fc1cc(Cl)c(-c2noc(C3CC3)c2CCl)c(Cl)c1]

Thionyl chloride (6.6 mL, 90 mmol) was added to a mixture of (5-cyclopropyl-3-(2,6-dichloro-4-fluorophenyl)isoxazol-4-yl)methanol (9.1 g, 30 mmol) in dichloromethane (150 mL) at room temperature. The mixture was then heated at about 45° C. for 45 minutes. The mixture was concentrated under reduced pressure. The residue was taken up in diethyl ether and re-concentrated. This was repeated two more times to provide the desired intermediate, which was carried forward without further purification LCMS-ESI⁺ (m/z): [M+H]⁺ calcd for C₁₃H₁₀Cl₃FNO: 319.97; found: 320.50.

Step 6: Preparation of 4-((4-bromo-3-chlorophenoxy)methyl)-5-cyclopropyl-3-(2,6-dichloro-4-fluorophenyl)isoxazole

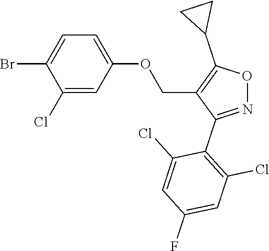 [SMILES: Fc1cc(Cl)c(-c2noc(C3CC3)c2COc2ccc(Br)c(Cl)c2)c(Cl)c1]

A solution of crude 4-(chloromethyl)-5-cyclopropyl-3-(2,6-dichloro-4-fluorophenyl)isoxazole (4.2 g, 13 mmol) in N,N-dimethylformamide (50 mL) was treated with 4-bromo-3-chlorophenol (2.7 g, 13 mmol), sodium iodide (3.3 g, 22 mmol), and anhydrous potassium carbonate (3.6 g, 26 mmol). The mixture was heated at about 60° C. for 25 minutes. After cooling, the mixture was filtered, and the filtrate was concentrated under reduced pressure. The residue was purified by flash chromatography (silica gel) to provide the desired intermediate. LCMS-ESI⁺ (m/z): [M+H]⁺ calcd for C₁₉H₁₃BrCl₃FNO₂: 489.91: found: 490.10.

Step 7: Preparation of tert-butyl 3-(2-chloro-4-((5-cyclopropyl-3-(2,6-dichloro-4-fluorophenyl)isoxazol-4-yl)methoxy)phenyl)-3-hydroxyazetidine-1-carboxylate

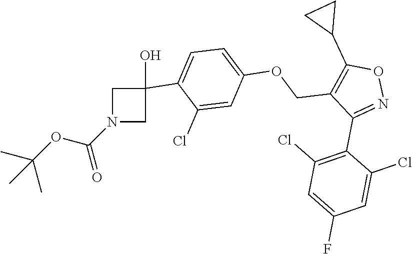 [SMILES: CC(C)(C)OC(=O)N1CC(O)(c2ccc(OCc3c(-c4c(Cl)cc(F)cc4Cl)noc3C3CC3)cc2Cl)C1]

Under an atmosphere of argon, a solution of 4-((4-bromo-3-chlorophenoxy)methyl)-5-cyclopropyl-3-(2,6-dichloro-4-fluorophenyl)isoxazole (5.1 g, 10 mmol) in tetrahydrofuran (27 mL) was treated with isopropylmagnesium chloride/lithium chloride solution (1.3 M, 12 mL, 15 mmol) via syringe. The resulting mixture was stirred for one hour before the introduction of an additional volume of isopropylmagnesium chloride/lithium chloride solution (1.3 M, 4.0 mL, 5.2 mmol). After the passage of 45 minutes of stirring, another portion of of isopropylmagnesium chloride/lithium chloride solution (1.3 M, 1.0 mL, 1.3 mmol) was added. After one hour of stirring, tert-butyl 3-oxoazetidine-1-carboxylate (3.8 g, 22 mmol) was added in a single portion to the Grignard mixture, which was being cooled in a wet ice/acetone bath. After 30 minutes of stirring, an additional portion of tert-butyl 3-oxoazetidine-1-carboxylate (0.80 g, 4.7 mmol) was added. The mixture was quenched with 10% aqueous citric acid solution (50 mL). The aqueous phase was extracted three times with ethyl acetate. Combined organics were washed once each with water and saturated aqueous sodium chloride solution, dried over anhydrous magnesium sulfate, filtered, and concentrated under reduced pressure. The residue was purified by flash chromatography (silica gel) to provide the desired intermediate. LCMS-ESI⁺ (m/z): [M+H]⁺ calcd for C₂₇H₂₇Cl₃FN₂O₅: 584.86; found: 483.19.

Step 8: Preparation of 3-(2-chloro-4-((5-cyclopropyl-3-(2,6-dichloro-4-fluorophenyl)isoxazol-4-yl)methoxy)phenyl)azetidin-3-ol tosylate salt

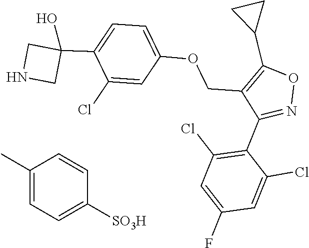 [SMILES: Cc1ccc(S(=O)(=O)O)cc1 ; OC1(c2ccc(OCc3c(-c4c(Cl)cc(F)cc4Cl)noc3C3CC3)cc2Cl)CNC1]

p-Toluenesulfonic acid monohydrate (3.5 g, 18 mmol) was added to a mixture of tert-butyl 3-(2-chloro-4-((5-cyclopropyl-3-(2,6-dichloro-4-fluorophenyl)isoxazol-4-yl)methoxy)phenyl)-3-hydroxyazetidine-1-carboxylate (5.3 g, 9.1 mmol) and isopropanol (21 mL). The mixture was heated overnight at about 50° C. and subsequently cooled in a wet ice/acetone bath with magnetic stirring. The resulting suspension was heated almost back to homogeneity at about 50° C. After the mixture cooled, hexane was added and the resulting suspension was sonicated and then warmed to about 50° C. The solid was collected by suction filtration, washed with hexane, and dried in an about 75° C. vacuum oven to provide the tosylate salt of the desired intermediate. LCMS-ESI⁺ (m/z): [M+H]⁺ calcd for C₂₂H₁₉C₁₃FN₂O₃: 483.04; found: 483.19.

Step 9: Preparation of methyl 6-(3-(2-chloro-4-((5-cyclopropyl-3-(2,6-dichloro-4-fluorophenyl)isoxazol-4-yl)methoxy)phenyl)-3-hydroxyazetidin-1-yl)nicotinate

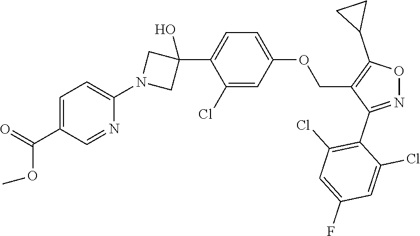 [SMILES: COC(=O)c1ccc(N2CC(O)(c3ccc(OCc4c(-c5c(Cl)cc(F)cc5Cl)noc4C4CC4)cc3Cl)C2)nc1]

A mixture of 3-(2-chloro-4-((5-cyclopropyl-3-(2,6-dichloro-4-fluorophenyl)isoxazol-4-yl)methoxy)phenyl)azetidin-3-ol tosylate (0.47 g, 0.71 mmol), methyl 6-chloronicotinate (0.15 g, 0.90 mmol), and potassium carbonate (0.49 g, 3.6 mmol) in N,N-dimethylformamide (4 mL) was heated overnight at 80° C. The mixture was partitioned between ethyl acetate and water. The aqueous phase was extracted three times with ethyl acetate. Combined extracts were washed successively with water and saturated aqueous sodium chloride solution, dried over anhydrous magnesium sulfate, filtered, and concentrated under reduced pressure. The residue was purified by flash chromatography (silica gel) to provide the desired intermediate. LCMS-ESI⁺ (m/z): [M+H]⁺ calcd for C₂₉H₂₄Cl₃FN₃O₅: 618.07; found: 618.41.

Step 10: Preparation of 6-(3-(2-chloro-4-((5-cyclopropyl-3-(2,6-dichloro-4-fluorophenyl)isoxazol-4-yl)methoxy)phenyl)-3-hydroxyazetidin-1-yl)nicotinic acid

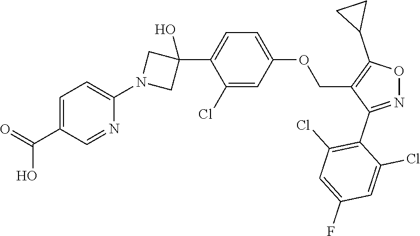 [SMILES: O=C(O)c1ccc(N2CC(O)(c3ccc(OCc4c(-c5c(Cl)cc(F)cc5Cl)noc4C4CC4)cc3Cl)C2)nc1]

A mixture of methyl 6-(3-(2-chloro-4-((5-cyclopropyl-3-(2,6-dichloro-4-fluorophenyl)isoxazol-4-yl)methoxy)phenyl)-3-hydroxyazetidin-1-yl)isonicotinate (0.32 g, 0.51 mmol) and lithium hydroxide monohydrate (43 mg, 1.0 mmol) in tetrahydrofuran/water (1:1, 10 mL) was stirred at room temperature overnight.

The mixture was concentrated under reduced pressure to remove most of the volatiles. The resulting aqueous mixture was further diluted with water and treated with acetic acid followed by 10% aqueous hydrochloric acid solution. The resulting precipitate was collected by suction filtration, washed with water, and dried in an about 50° C. vacuum oven to provide the desired material as the free acid. LCMS-ESI⁺ (m/z): [M+H]⁺ calcd for C₂₈H₂₂Cl₃FN₃O₅: 604.05: found: 604.41.

Step 11: Preparation of 6-(3-(2-chloro-4-((5-cyclopropyl-3-(2,6-dichloro-4-fluorophenyl)isoxazol-4-yl)methoxy)phenyl)-3-hydroxyazetidin-1-yl)nicotinic acid, tris salt

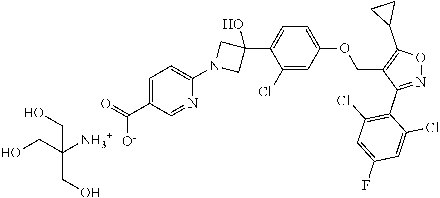 [SMILES: [NH3+]C(CO)(CO)CO ; O=C([O-])c1ccc(N2CC(O)(c3ccc(OCc4c(-c5c(Cl)cc(F)cc5Cl)noc4C4CC4)cc3Cl)C2)nc1]

6-(3-(2-chloro-4-((5-cyclopropyl-3-(2,6-dichloro-4-fluorophenyl)isoxazol-4-yl)methoxy)phenyl)-3-hydroxyazetidin-1-yl)nicotinic acid (0.29 g, 0.48 mmol) was taken up as a suspension in MeOH/water (95:5, 2 mL), treated with tromethamine (58 mg, 0.48 mmol), heated at 55° C., and concentrated to provide the desired acid as the tromethamine salt. ¹H NMR (400 MHZ, Methanol-d₄) δ 8.65 (dd, J=2.2, 0.8 Hz, 1H), 8.05 (dd, J=8.7, 2.2 Hz, 1H), 7.38 (d, J=8.3 Hz, 2H), 7.35 (d, J=8.7 Hz, 1H), 6.87 (d, J=2.6 Hz, 1H), 6.78 (dd, J=8.6, 2.6 Hz, 1H), 6.42 (dd, J=8.8, 0.8 Hz, 1H), 4.92 (s, 2H), 4.57 (dd, J=9.3, 1.1 Hz, 2H), 4.29 (dd, J=9.2, 1.1 Hz, 2H), 3.63 (s, 7H), 2.32 (tt, J=8.0, 5.5 Hz, 1H), 1.25-1.11 (m, 4H).

### Example 2: Synthesis of Comparative Example 1

### 6-(3-(2-chloro-4-((5-cyclopropyl-3-(2,6-dichloro-4-fluorophenyl)isoxazol-4-yl)methoxy)phenyl)-3-hydroxyazetidin-1-yl)-5-fluoronicotinic acid

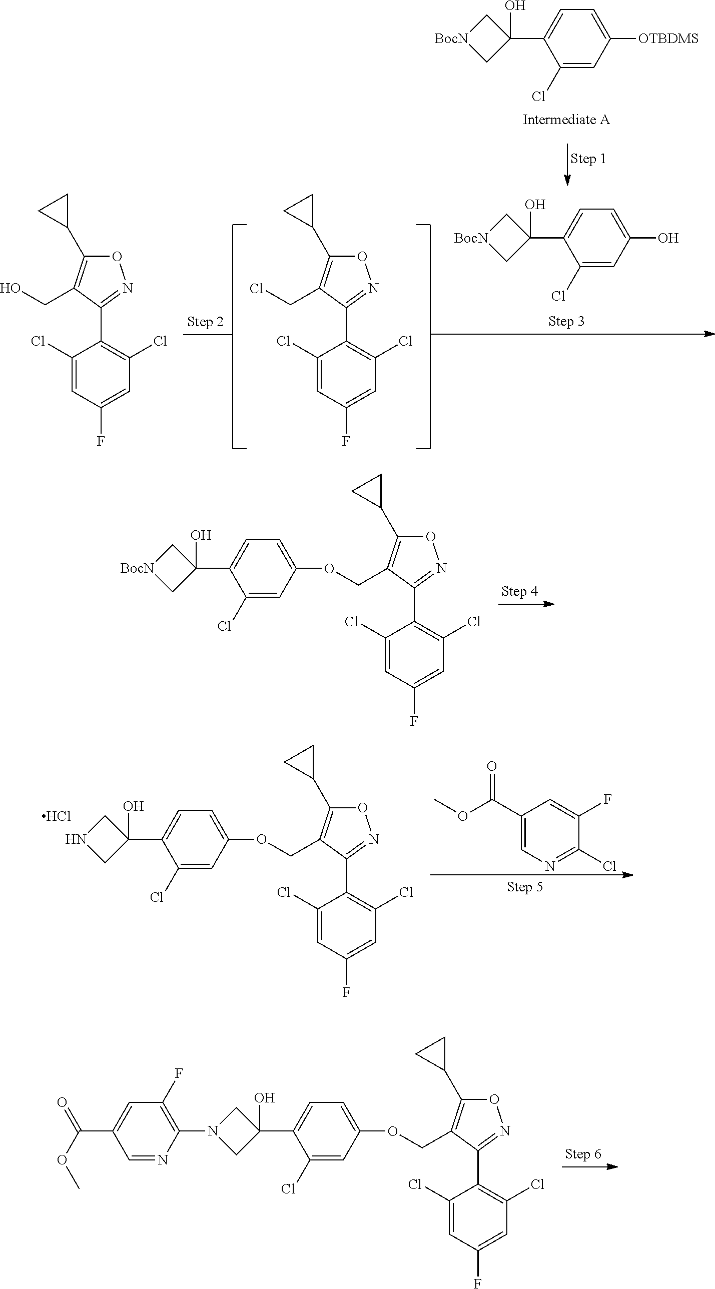

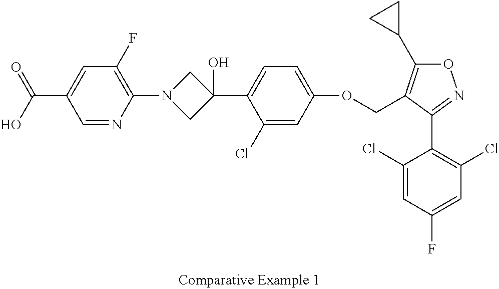 [SMILES: O=C(O)c1cnc(N2CC(O)(c3ccc(OCc4c(-c5c(Cl)cc(F)cc5Cl)noc4C4CC4)cc3Cl)C2)c(F)c1]

### Synthesis of Intermediate A:

To a solution of (4-bromo-3-chlorophenoxy)(tert-butyl)dimethylsilane (60 g. 187 mmol) in THF (500 mL) was added dropwise n-BuLi (2.5 M, 75 mL) at about −78° C. under N₂. The reaction was stirred at about −78° C. for 1 hour. Next a solution of tert-butyl 3-oxoazetidine-1-carboxylate (27 g. 155 mmol) in THF (500 mL) was added dropwise to the mixture at −78° C. Then the reaction was stirred at about 20° C. for 3 hours. The reaction mixture was poured into H₂O (1 L) and extracted with EtOAc (2 L) three times. The combined organic layers were washed with water (1 L), dried over Na₂SO₄, filtered and concentrated in vacuo. The crude product was purified by silica gel chromatography eluted with 10:1 petroleum ether: EtOAc to give 3-(4-((tert butyldimethylsilyl)oxy)-2-chlorophenyl)azetidin-3-ol (Intermediate A).

Step 1: tert-butyl 3-(2-chloro-4-hydroxyphenyl)-3-hydroxyazetidine-1-carboxylate

To a solution of tert-butyl 3-(4-((tert-butyldimethylsilyl)oxy)-2-chlorophenyl)-3-hydroxyazetidine-1-carboxylate (Intermediate A, 1.27 g, 3.07 mmol) in THF (50.0 mL) at about −10° C. was added 1M TBAF in THF (3.68 mL, 3.68 mmol) dropwise. The reaction was stirred for 2 hours and was concentrated to afford tert-butyl 3-(2-chloro-4-hydroxyphenyl)-3-hydroxyazetidine-1-carboxylate, which was used without further purification.

Step 2: 4-(chloromethyl)-5-cyclopropyl-3-(2,6-dichloro-4-fluorophenyl)isoxazole

A solution of (5-cyclopropyl-3-(2,6-dichloro-4-fluorophenyl)isoxazol-4-yl)methanol (45 mg. 2.80 mmol) in DCM (28.0 mL) was cooled to about 0° C. Thionyl chloride (1.02 mL, 14.0 mmol) was added and the solution was heated at about 45° C. for 1 hour. The reaction was concentrated to dryness and used without purification in the next step.

Step 3: tert-butyl 3-(2-chloro-4-((5-cyclopropyl-3-(2,6-dichloro-4-fluorophenyl)isoxazol-4-yl)methoxy)phenyl)-3-hydroxyazetidine-1-carboxylate

A solution of tert-butyl 3-(2-chloro-4-hydroxyphenyl)-3-hydroxyazetidine-1-carboxylate (922 mg. 3.07 mmol) in DMF (28.0 mL) was added to crude 4-(chloromethyl)-5-cyclopropyl-3-(2,6-dichloro-4-fluorophenyl)isoxazole, followed by the addition of potassium carbonate (773 mg, 5.60 mmol). The mixture was heated at about 60° C. for 8 hours. The reaction was concentrated, diluted with water and extracted with EtOAc (3×). The combined organic layers were washed with water, brine, dried over MgSO₄, filtered and concentrated. The crude product was purified by silica gel chromatography (DCM/Et₂O/MeOH) to afford tert-butyl 3-(2-chloro-4-((5-cyclopropyl-3-(2,6-dichloro-4-fluorophenyl)isoxazol-4-yl)methoxy)phenyl)-3-hydroxyazetidine-1-carboxylate. LCMS-ESI⁺ (m/z): [(M+H)-BOC]⁺ calcd for calcd 483.04: found 483.04.

Step 4: 3-(2-chloro-4-((5-cyclopropyl-3-(2,6-dichloro-4-fluorophenyl)isoxazol-4-yl)methoxy)phenyl)azetidin-3-ol

To a solution of tert-butyl 3-(2-chloro-4-((5-cyclopropyl-3-(2,6-dichloro-4-fluorophenyl)isoxazol-4-yl)methoxy)phenyl)-3-hydroxyazetidine-1-carboxylate (1.52 g, 2.60 mmol) in DCM (130 mL) was added 4 N HCl in 1,4-dioxane (26.0 mL, 104 mmol). The solution was stirred at room temperature for 2.5 hours and was concentrated to dryness to afford 3-(2-chloro-4-((5-cyclopropyl-3-(2,6-dichloro-4-fluorophenyl)isoxazol-4-yl)methoxy)phenyl)azetidin-3-ol as the hydrochloride salt, which was used without further purification. LCMS-ESI⁺ (m/z): [M+H]⁺ calcd 483.04; found 483.03.

Step 5: methyl 6-(3-(2-chloro-4-((5-cyclopropyl-3-(2,6-dichloro-4-fluorophenyl)isoxazol-4-yl)methoxy)phenyl)-3-hydroxyazetidin-1-yl)-5-fluoronicotinate

A mixture of methyl 6-chloro-5-fluoropyridine (235 mg, 1.24 mmol), 3-(2-chloro-4-((5-cyclopropyl-3-(2,6-dichloro-4-fluorophenyl)isoxazol-4-yl)methoxy)phenyl)azetidin-3-ol as the hydrochloride salt (495 mg, 0.952 mmol) and potassium carbonate (1.05 g, 7.61 mmol) in DMF (30.0 mL) was heated at about 60° C. for 1 hour. The reaction was concentrated, diluted with water and extracted with EtOAc (3×). The combined organic layers were washed with brine, dried over MgSO₄, filtered and concentrated. The crude mixture was purified by silica gel chromatography (DCM/Et₂O/MeOH) to afford methyl 6-(3-(2-chloro-4-((5-cyclopropyl-3-(2,6-dichloro-4-fluorophenyl)isoxazol-4-yl)methoxy)phenyl)-3-hydroxyazetidin-1-yl)-5-fluoronicotinate. LCMS-ESI⁺ (m/z): [M+H]⁺ calcd 636.07; found 635.96.

Step 6: 6-(3-(2-chloro-4-((5-cyclopropyl-3-(2,6-dichloro-4-fluorophenyl)isoxazol-4-yl)methoxy)phenyl)-3-hydroxyazetidin-1-yl)-5-fluoronicotinic acid (Comparative Example 1)

To a solution of methyl 6-(3-(2-chloro-4-((5-cyclopropyl-3-(2,6-dichloro-4-fluorophenyl)isoxazol-4-yl)methoxy)phenyl)-3-hydroxyazetidin-1-yl)-5-fluoronicotinate (364 mg, 0.571 mmol) in THF/water (1:1, 20.0 mL) was added lithium hydroxide monohydrate (41.3 mg. 0.984 mmol). The solution was stirred for 18 hours, concentrated to remove THF and diluted with water (10.0 mL). The pH was adjusted to 3 using 1N HCl. The solids were filtered, washed with water, dissolved in ACN/water and lyophilized to afford 6-(3-(2-chloro-4-((5-cyclopropyl-3-(2,6-dichloro-4-fluorophenyl)isoxazol-4-yl)methoxy)phenyl)-3-hydroxyazetidin-1-yl)-5-fluoronicotinic acid (Comparative Example 1). LCMS-ESI⁺ (m/z): [M+H]⁺ calcd 622.05: found 622.12. ¹H NMR (400 MHZ, DMSO-d₆) δ 12.84 (bs, 1H), 8.44 (t, J=1.7 Hz, 1H), 7.79-7.63 (m, 3H), 7.39 (d, J=8.7 Hz, 1H), 6.95 (d, J=2.5 Hz, 1H), 6.77 (dd, J=8.6, 2.6 Hz, 1H), 6.28 (s, 1H), 4.93 (s, 2H), 4.70 (d, J=9.8 Hz, 2H), 4.34 (d, J=9.5 Hz, 2H), 2.50-2.43 (m, 1H), 1.22-1.08 (m, 4H).

### Example 3: FRET Activity Assay

Determination of a ligand mediated cofactor peptide interaction to quantify ligand binding to the nuclear receptor FXR was performed as follows.

Preparation of human FXR alpha ligand binding domain (LBD): The human FXRalpha LBD was expressed in E. coli strain BL21 (DE3) as an N-terminally GST tagged fusion protein. The DNA encoding the FXR ligand binding domain was cloned into vector pDEST15 (Invitrogen). Expression was under control of an IPTG inducible T7 promoter. The amino acid boundaries of the ligand binding domain were amino acids 187-472 of Database entry NM_005123 (RefSeq). Expression and purification of the FXR-LBD: An overnight preculture of a transformed E.coli strain was diluted 1:20 in LB-Ampicillin medium and grown at 30° C. to an optical density of OD₆₀₀₌0.4-0.6. Gene expression was then induced by addition of 0.5 mM IPTG. Cells were incubated an additional 6 h at 30° C., 180 rpm. Cells were collected by centrifugation (7000×g. 7 minutes, room temperature). Per liter of original cell culture, cells were resuspended in 10 mL lysis buffer (50 mM Glucose, 50 mM Tris pH 7.9, 1 mM EDTA and 4 mg/mL lysozyme) and left on ice for 30 min. Cells were then subjected to sonication and cell debris was removed via centrifugation (22000×g. 30 min. 4° C.). 0.5 mL prewashed Glutathione 4B sepharose slurry (Qiagen) was added per 10 mL of supernatant and the suspension was kept slowly rotating for 1 hour at 4° C. Glutathione 4B sepharose beads were pelleted by centrifugation (2000×g, 15 seconds. 4° C.) and washed twice in wash buffer (25 mM Tris, 50 mM KCl, 4 mM MgCl₂ and 1M NaCl). The pellet was resuspended in 3 mL elution buffer per liter of original culture (elution buffer: 20 mM Tris, 60 mM KCl, 5 mM MgCl₂ and 80 mM glutathione added immediately prior to use as powder). The suspension was left rotating for 15 min at 4° C. the beads pelleted and eluted again with half the volume of elution buffer than the first time. The eluates were pooled and dialysed overnight in 20 mM Hepes buffer (pH 7.5) containing 60 mM KCl, 5 mM MgCl₂ as well as 1 mM dithiothreitol and 10% (v/v) glycerol. The protein was analysed by SDS-Page.

The method measures the ability of putative ligands to modulate the interaction between the purified bacterially expressed FXR ligand binding domain (LBD) and a synthetic biotinylated peptide based on residues 676-700 of SRC-1 (LCD2, 676-700). The sequence of the peptide used was B-CPSSHSSLTERHKILHRLLQEGSPS-COOH (SEQ ID NO: 1) where the N-terminus was biotinylated (B). The ligand binding domain (LBD) of FXR was expressed as fusion protein with GST in BL-21 cells using the vector pDEST15. Cells were lysed by sonication, and the fusion proteins purified over glutathione sepharose (Pharmacia) according to the manufacturers instructions. For screening of compounds for their influence on the FXR-peptide interaction. the Perkin Elmer LANCE technology was applied. This method relies on the binding dependent energy transfer from a donor to an acceptor fluorophor attached to the binding partner of interest. For ease of handling and reduction of background from compound fluorescence LANCE technology makes use of generic fluorophore labels and time resolved detection Assays were done in a final volume of 25 μL in a 384 well plate, in a Tris-based buffer (20 mM Tris-HCl pH 7.5; 60 mM KCl, 5 mM MgCl₂: 35 ng/μL BSA), containing 20-60 ng/well recombinantly expressed FXR-LBD fused to GST, 200-600 nM N-terminally biotinylated peptide. representing SRC1 aminoacids 676-700, 200 ng/well Streptavidin-xlAPC conjugate (Prozyme) and 6-10 ng/well Eu W1024-antiGST (Perkin Elmer). DMSO content of the samples was kept at 1%. After generation of the assay mix and diluting the potentially FXR modulating ligands. the assay was equilibrated for 1 hour in the dark at room temperature in FIA-plates black 384 well (Greiner). The LANCE signal was detected by a Perkin Elmer VICTOR2VTM Multilabel Counter. The results were visualized by plotting the ratio between the emitted light at 665 and 615 nm. A basal level of FXR-peptide formation is observed in the absence of added ligand. Ligands that promote the complex formation induce a concentration-dependent increase in time-resolved fluorescent signal. Compounds which bind equally well to both monomeric FXR and to the FXR-peptide complex would be expected to give no change in signal, whereas ligands which bind preferentially to the monomeric receptor would be expected to induce a concentration-dependent decrease in the observed signal.

To assess the agonistic potential of the compounds, EC₅₀ values were determined for example compounds and are listed below in Table 1 (FRET EC₅₀). As indicated in Table 1, the compound of Example I was assessed along with Comparative Example 1, and Comparative Example 2 (Example 3 of U.S. Patent Application Publication No. 2017/0355685), the chemical structures of which are depicted in the table below.

### Example 4: Mammalian One Hybrid (M1H) Assay

Determination of a ligand mediated Gal4 promoter driven transactivation to quantify ligand binding mediated activation of FXR was performed as follows.

The cDNA part encoding the FXR ligand binding domain was cloned into vector pCMV-BD (Stratagene) as a fusion to the yeast GAL4 DNA binding domain under the control of the CMV promoter. The amino acid boundaries of the ligand binding domain were amino acids 187-472 of Database entry NM_005123 (RefSeq). The plasmid pFR-Luc (Stratagene) was used as the reporter plasmid, containing a synthetic promoter with five tandem repeats of the yeast GAL4 binding sites, driving the expression of the Photinus pyralis (American firefly) luciferase gene as the reporter gene. In order to improve experimental accuracy the plasmid pRL-CMV (Promega) was cotransfected. pRL-CMV contains the constitutive CMV promoter, controlling the expression of the Renilla reniformis luciferase. All Gal4 reporter gene assays were done in HEK293 cells (obtained from DSMZ, Braunschweig, Germany) grown in MEM with L-Glutamine and Earle's BSS supplemented with 10% fetal bovine serum, 0.1 mM nonessential amino acids, 1 mM sodium pyruvate, and 100 units Penicilin/Streptavidin per mL at about 37° C. in 5% CO₂. Medium and supplements were obtained from Invitrogen. For the assay, 5×10⁵ cells were plated per well in 96 well plates in 100 μL per well MEM without Phenol Red and L-Glutamine and with Earle's BSS supplemented with 10% charcoal/dextran treated FBS (HyClone, South Logan, Utah), 0.1 mM nonessential amino acids, 2 mM glutamine, 1 mM sodium pyruvate, and 100 units Penicilin/Streptavidin per mL, incubated at about 37° C. in 5% CO₂. The following day the cells were >90% confluence. Medium was removed and cells were transiently transfected using 20 μL per well of an OptiMEM—polyethylene-imine-based transfection-reagent (OptiMEM, Invitrogen; Polyethyleneimine, Aldrich Cat No. 40,827-7) including the three plasmids described above. MEM with the same composition as used for plating cells was added 2-4 hours after addition of transfection mixture. Then compound stocks, prediluted in MEM were added (final vehicle concentration not exceeding 0.1%). Cells were incubated for additional 16 hours before firefly and renilla luciferase activities were measured sequentially in the same cell extract using a Dual-Light-Luciferase-Assay system (Dyer et al., Anal. Biochem. 2000, 282, 158-161). All experiments were done in triplicate.

To assess the FXR agonistic potency of the example compounds, potency was determined in the MIH assay and is listed below in Table 1 (MIH EC₅₀).

TABLE 1

| | | FRET EC₅₀ | M1H EC₅₀ |
|---|---|---|---|
| Example | | (nM) | (nM) |
| Example 1 | 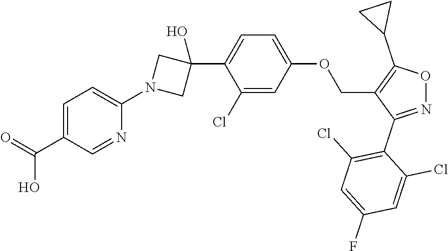 [SMILES: O=C(O)c1ccc(N2CC(O)(c3ccc(OCc4c(-c5c(Cl)cc(F)cc5Cl)noc4C4CC4)cc3Cl)C2)nc1] | 14.2 | 5.5 |
| Comparative Example 1 | 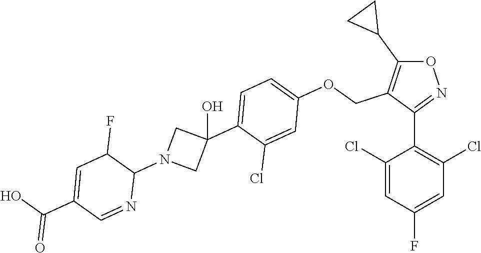 [SMILES: O=C(O)C1=CC(F)C(N2CC(O)(c3ccc(OCc4c(-c5c(Cl)cc(F)cc5Cl)noc4C4CC4)cc3Cl)C2)N=C1] | 7.5 | 1.2 |
| Comparative Example 2 | 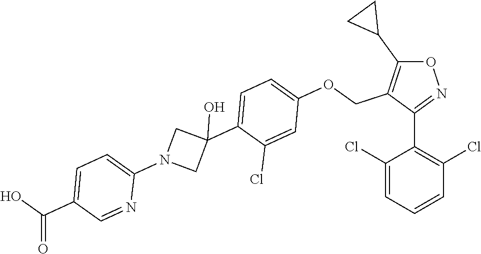 [SMILES: O=C(O)c1ccc(N2CC(O)(c3ccc(OCc4c(-c5c(Cl)cccc5Cl)noc4C4CC4)cc3Cl)C2)nc1] | 3.4 | 2 |

### Example 5: Assessment of In Vivo Pharmacodynamics in Cynomolgus Monkey

In vivo pharmacodynamics of Formula (I) of Example 1, Comparative Example 1, and Comparative Example 2 were determined as follows.

### Test Article and Formulation

### Oral Suspension Doses

Oral solution doses of Formula (I) (Example 1) were formulated at concentrations of 1 mg/mL in aqueous suspensions of 1% sodium laurel sulfate (SLS), 1% ethanol, and 98% water at pH 2. Oral suspension doses of Comparative Example 1, and Comparative Example 2 were formulated at concentrations of 2.5 mg/mL in 1% hydroxypropyl methylcellulose (HPMC), 0.5% Tween 80, and 98.5% water.

### Intravenous Doses

Intravenous doses of compounds of Formula (I) (Example 1), Comparative Example 1, and Comparative Example 2 were formulated at concentrations of 0.5 mg/mL in a vehicle including 5% DMSO, 15% NMP, 60% PEG 300 and water with 1.1 equivalents of NaOH.

### Animals

Each dosing group consisted of three to six male Cynomolgus monkeys. At dosing, the animals weighed between 2.4 and 4.4 kg.

### Dosing

The oral test articles were administered to the monkeys via oral gavage at 2 mL/kg or nasogastric untubation at 5 mL/kg for a dose of 5 mg/kg. The intravenous test articles were administered as an approximately 30-minute infusion via an indwelling catheter in a saphenous or cephalic vein at approximately 2 mL/kg for a dose of 1 mg/kg.

### Sample Collection

Venous blood samples were taken at specified time points after dosing from each animal. The blood samples were collected and transferred into tubes containing potassium (K₂) EDTA anticoagulant.

### Determination of FGF19 Concentrations in Plasma

The FGF19 ELISA assay kit from BioVendor (product number RD191107200R) was used to determine FGF19 concentrations in the collected blood samples.

### Determination of Drug Concentration in Plasma

For analysis on an API 5000 LC/MS/MS system, an aliquot of 50 μL of each plasma sample was treated with 200 μL of acetonitrile (ACN) containing internal standard. The above solution was centrifuged at 5000 RPM for 10 minutes and 50 μL of supernatant was transferred to a clean 96-well plate, followed by the addition of 200 μL of water. An aliquot of 10 μL was injected to the API 5000 LC/MS/MS system.

For analysis on an Applied Biosystems API 5500 LC/MS/MS system, an aliquot of 20 μL of each plasma sample was treated with 120 μL acetonitrile (ACN) containing internal standard. The above solution was centrifuged and 100 μL of supernatant was transferred to a clean 96-well plate, followed by the addition of 100 μL of water. An aliquot of 7-10 μL was injected to the API 5500 LC/MS/MS system.

### HPLC Conditions

A Zorbax Extend C18 HPLC column (50×2.1 mm, 3.5 μm) from Agilent

Technologies (Part #735700-902) (Comparative Example 1, and Comparative Example 2) or a Waters BEH C18 column (50×2.1 mm, 1.7 μm) (Example 1) was used. For the Zorbax Extend C18 HPLC coumn, mobile phase A contained an aqueous solution of 1% acetonitrile in 10 mM ammonium formate adjusted to pH 3.0 with formic acid and mobile phase B contained and 10% 10 mM ammonium formate in acetonitrile adjusted to pH 4.6 with formic acid. For the Waters BEH C18 column, mobile phase A contained 95% water, 5% acetonitrile, and 0.1% formic acid and mobile phase B contained 50% methanol, 50% acetonitrile, and 0.1% formic acid. A Thermo Aria multiplexer with two identical Agilent 1200 series binary pumps (P/N G1312A Bin Pump) was used for elution and separation. The elution programs used are set forth in the following Table 2 for the Zorbax Extend C18 HPLC and Table 3 for the Waters BEH C18 column.

TABLE 2

| Time | | Flow Rate | Mobile | Mobile |
|---|---|---|---|---|
| (sec) | Step Comments | (mL/min) | Phase A (%) | Phase B (%) |
| 30 | Sample Loading | 0.50 | 85 | 15 |
| 180 | Ramp | 0.50 | 50 | 50 |
| 90 | Ramp | 0.50 | 99 | 1 |
| 60 | Elution | 0.50 | 99 | 1 |
| 120 | Re-equilibrium | 0.50 | 85 | 15 |

TABLE 3

| Time | | Flow Rate | Mobile | Mobile |
|---|---|---|---|---|
| (sec) | Step Comments | (mL/min) | Phase A (%) | Phase B (%) |
| 0 | Sample Loading | 0.80 | 45 | 55 |
| 60 | Ramp | 0.80 | 35 | 65 |
| 63 | Ramp | 0.80 | 5 | 95 |
| 78 | Ramp | 0.80 | 5 | 95 |
| 90 | Step | 0.80 | 45 | 55 |

An API 5000 triple quadrupole mass spectrometer from AB Sciex, Foster City, CA was used in multiple reaction monitoring mode to quantify the compounds. The mass spectrometry parameters used are set forth in the following Table 4.

TABLE 4

| | Spray | | | Collision | Dryer |
|---|---|---|---|---|---|
| Ion | voltage | Gas 1 | Gas 2 | gas | temperature |
| source | (V) | (Arb) | (Arb) | (Arb) | (° C.) |
| Turbo Ion | 5500 | 70 | 50 | 6 | 550 |
| Spray | | | | | |

### Oral Versus IV Administration in Cynomolgus Monkeys

Activation of the Farnesoid X receptor in the distal small intestine as well as systemically in organs such as the liver, directly causes the expression and secretion of FGF19. Plasma FGF 19 levels were evaluated as a pharmacodynamics marker of FXR activation in male cynomolgus monkeys following administration of oral and intravenous administration of doses of Example 1, Comparative Example 1, or Comparative Example 2. The difference between levels of FGF 19 following oral administration (PO) versus intravenous administration (IV) were used to compare the degree of intestinal FXR agonism (via analysis of PO data) and systemic FXR agonism (via analysis of IV data) for each compound administered. Plasma levels of drug and FGF19 were measured at various time points over a 24 hour period to generate pharmacokinetic and pharmacodynamics curves.

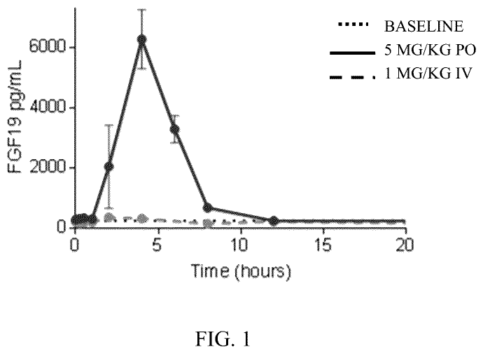

FIG. 1 depicts a chart of FGF19 plasma concentration over time in cynomolgus monkey after dosing with the compound of Example 1.

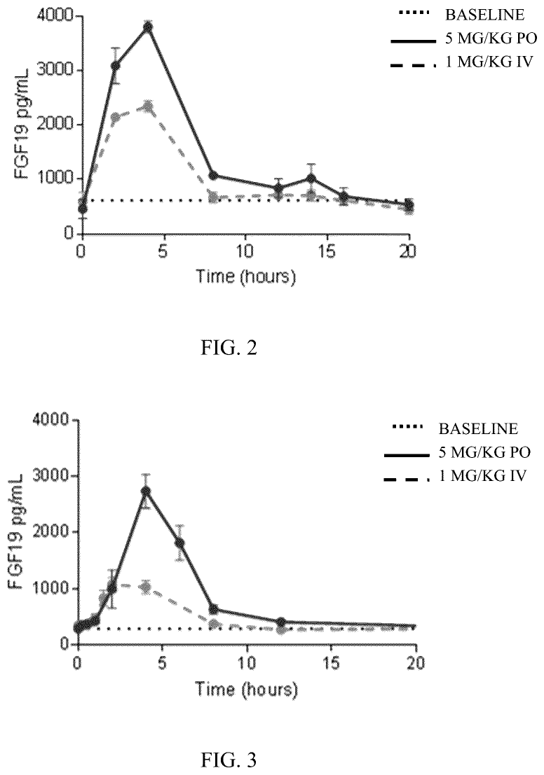

FIG. 2 depicts a chart of FGF19 plasma concentration over time in cynomolgus monkey after dosing with the compound of Comparative Example 1.

FIG. 3 depicts a chart of FGF19 plasma concentration over time in cynomolgus monkey after dosing with the compound of Comparative Example 2.

Pharmacokinetic data are summarized in Table 5 below.

TABLE 5

| | | | FGF19 | | Ratio | |
|---|---|---|---|---|---|---|
| | | FGF19 | AUCₗₐₛₜ | GS-AUCₗₐₛₜ | AUCₗₐₛₜ to | |
| Example | Group | Cₘₐₓ(pg/mL) | (pg/mL.h) | (nM.h) | FG19/GS | PO/IV |
| Example 1 | IV | 432 +/− 100 | 5144 +/− 2199 | 3150 +/− 1030 | 1.6 | 7.8 |
| | PO | 6260 +/− 2194 | 28153 +/− 11539 | 2250 +/− 1640 | 12.5 | |
| Comparative | IV | 2350 +/− 179 | 22798 +/− 2387 | 1630 +/− 323 | 14 | 1.37 |
| Example 1 | PO | 3810 +/− 195 | 32085 +/− 4252 | 1670 +/− 250 | 19.2 | |
| Comparative | IV | 1179 +/− 226 | 10757 +/− 2551 | 3140 +/− 167 | 3.4 | 8.2 |
| Example 2 | PO | 3186 +/− 856 | 18193 +/− 7134 | 643 +/− 216 | 28 | |

As is reflected in the results, the oral administration of Example 1, Comparative Example 1, and Comparative Example 2 all caused increases in plasma FGF19 over the dosing interval compared to baseline levels of FGF19 before dosing. This indicates agonism of the intestinal FXR receptor. The IV administration of Example I did not cause in increase in FGF19, indicating a lack of systemic FXR agonism. In contrast, IV administration of Comparative Examples 1 and 2 increased FGF19 levels compared to baseline levels of FGF19 before dosing, indicating systemic FXR agonism occurred.

These data suggest that ileal exposures of Example land Comparative Example 2 cause FXR activation in the intestinal epithelium. The data further suggest that systemic exposures of Comparative Example 1 and Comparative Example 2, through IV administration, causes greater systemic, non-intestinal FXR activation relative to Example 1.

### Example 6: UGT1A1 Inhibition

The compound of Formula (I) (Example 1) was tested for inhibition of UGT1A1 and was compared against Comparative Example 1. To perform the assay, sample compound was incubated with human UGT1A1 expressed Supersomes™ (0.25 mg/mL), alamethicin (25 μg/mg) and UDPGA (5 mM) in the presence of probe substrate estradiol (10 μM) for 30 mins at 37° C. A selective UGT1A1 inhibitor, atazanavir, was screened alongside the test compounds as a positive control. The reactions were terminated by quenching an aliquot into two volumes of methanol. The samples were centrifuged at 2500 rpm for 30 mins at 4° C., and aliquots of the supernatant were diluted with formic acid in deionised water (final formic acid concentration 0.1%) and internal standard. Cyprotex generic LC-MS/MS conditions were used to monitor estradiol 3-glucuronide formation. A decrease in the formation of the metabolite compared to vehicle control is used to calculate an IC50 value.

The data showed that the compound of Example 1 showed the most potent UGT1A1 inhibition of the compounds compared. Example 1 had an IC₅₀ of 0.44 μM.

### Example 7: CYP2C8, CYP3A4, and CYP2B6 Inhibition

The compound of Formula (I) (Example 1) was assessed for CYP2C8, CYP3A4, and CYP2B6 inhibition and was compared against Comparative Example 1.

### CYP2C8

Six sample compound concentrations (0.1, 0.25, 1, 2.5, 10, 25 μM in DMSO; final DMSO concentration 0.25%) were incubated with human liver microsomes (1 mg/mL) and NADPH (1 mM) in the presence of the probe substrate tolbutamide (120 μM) for 60 minutes at 37° C. The selective CYP2C9 inhibitor, sulphaphenazole, was screened alongside the test compounds as a positive control.

### CYP3A4

Six sample compound concentrations (0.1, 0.25, 1, 2.5, 10, 25 μM in DMSO; final DMSO concentration 0.26%) were incubated with human liver microsomes (0.1 mg/mL) and NADPH (1 mM) in the presence of the probe substrate midazolam (2.5 μM) for 5 min at 37° C. The selective CYP3A4 inhibitor, ketoconazole, was screened alongside the test compounds as a positive control. Alternatively, six sample compound concentrations (0.1, 0.25, 1, 2.5, 10, 25 μM in DMSO: final DMSO concentration 0.275%) were incubated with human liver microsomes (0.5 mg/mL) and NADPH (1 mM) in the presence of the probe substrate testosterone (50 μM) for 5 min at 37° C. The selective CYP3A4 inhibitor, ketoconazole, was screened alongside the test compounds as a positive control.

### CYP2B6

Six test compound concentrations (0.1, 0.25, 1, 2.5, 10, 25 μM in DMSO; final DMSO concentration 0.3%) are incubated with human liver microsomes (0.1 mg/mL) and NADPH (1 mM) in the presence of the probe substrate bupropion (110 μM) for 5 min at 37° C. The selective CYP2B6 inhibitor, ticlopidine, is screened alongside the test compounds as a positive control.

For each of the CYP2B6, CYP2C8, and CYP3A4 incubations, the reactions were terminated by methanol addition. The samples were then centrifuged and analyzed by LC-MS/MS. Formic acid in deionised water (final concentration 0.1%) containing internal standard was added to the final sample prior to analysis. A decrease in the formation of the metabolites compared to vehicle control was used to calculate IC50.

The data showed that the compound of Example I did not inhibit CYP2B6, while Comparative Example 1 inhibited CYP2B6.

### Example 8: Preparation of Formula (I) mesylate Form I

Formula (I) mesylate Form I was prepared by combining 6-(3-(2-chloro-4-((5-cyclopropyl-3-(2,6-dichloro-4-fluorophenyl)isoxazol-4-yl)methoxy)phenyl)-3-hydroxyazetidin-1-yl)nicotinic acid (0.050 g, 0.0827 mmol) with methanesulfonic acid (8 uL, 0.123 mmol) in a vial with isopropyl alcohol (1 mL) resulting in a slurry. The slurry was heated to about 50° C. for about 30 minutes, then allowed to slowly cool to room temperature and slurry for about 16 hours. The slurry was then filtered and the isolated solids were characterized by XRPD presented in FIG. 4. The solids were then dried in a vacuum oven at about 50° C. and characterized by XRPD which resulted in the same pattern. Formula (I) mesylate Form I XRPD peaks included peaks at: 3.2, 6.4, 9.6, 12.8, 19.3, 22.1, 22.6, 25.8, 29.1° 2θ. XRPD peaks obtained are listed in Table 1 below.

TABLE 1

| | | Rel. |
|---|---|---|
| | | Int. |
| No. | Pos. [°2θ] | [%] |
| 1 | 3.2 | 55 |
| 2 | 6.4 | 24 |
| 3 | 8.0 | 4 |
| 4 | 9.6 | 78 |
| 5 | 10.6 | 3 |
| 6 | 12.8 | 46 |
| 7 | 17.3 | 4 |
| 8 | 18.2 | 7 |
| 9 | 19.3 | 59 |
| 10 | 20.1 | 2 |
| 11 | 22.1 | 23 |
| 12 | 22.6 | 100 |
| 13 | 23.7 | 6 |
| 14 | 24.3 | 10 |
| 15 | 25.8 | 20 |
| 16 | 26.5 | 3 |
| 17 | 27.3 | 5 |
| 18 | 28.1 | 3 |
| 19 | 29.1 | 19 |
| 20 | 32.5 | 12 |
| 21 | 34.4 | 3 |
| 22 | 35.5 | 2 |
| 23 | 36.9 | 9 |

DSC and TGA analyses were performed. The DSC thermogram of Formula (I) mesylate Form I showed an endothermic event at about 221° C. followed by an exothermic event, as is seen in FIG. 5. The TGA thermogram of Formula (I) mesylate Form I showed a weight loss of about 0.6% upon heating to about 200° C., as is seen in FIG. 6.

Formula (I) mesylate Form I, was also formed by taking up 6-(3-(2-chloro-4-((5-cyclopropyl-3-(2,6-dichloro-4-fluorophenyl)isoxazol-4-yl)methoxy)phenyl)-3-hydroxyazetidin-1-yl)nicotinic acid (3.00 g, 4.96 mmol) as a suspension in MeCN/water (1.5:1 v/v, 15 mL) and treating with an aqueous solution of sodium hydroxide (30 wt %, 0.56 mL, 5.95 mmol). The resulting solution was then charged over several hours to a second vessel containing an agitated solution of methane sulfonic acid (1.0 mL, 15.9 mmol) in MeCN (15 mL) preheated to about 50° C. The resulting slurry was aged at about 50° C. for several hours and then cooled to about 20° C. The slurry is filtered under vacuum and the resulting solids were dried under vacuum at elevated temperature up to about 60° C. to provide Formula (I) mesylate Form I.

Formula (I) mesylate form I was also formed by taking up 6-(3-(2-chloro-4-((5-cyclopropyl-3-(2,6-dichloro-4-fluorophenyl)isoxazol-4-yl)methoxy)phenyl)-3-hydroxyazetidin-1-yl)nicotinic acid (0.5 g, 0.83 mmol) as a suspension in acetone/water (97:3 v/v, 10 mL) and heating to about 55° C. Methane sulfonic acid (60 μL, 0.91 mmol) was charged to the slurry and the mixture was aged at about 55° C. for several hours. The slurry was cooled to about 20° C. and filtered under vacuum. The resulting solids were washed with acetone and dried under vacuum at elevated temperature up to about 60° C. to provide Formula (I) mesylate Form I.

Unless otherwise defined, all technical and scientific terms used herein have the same meaning as commonly understood by one of ordinary skill in the art to which this disclosure belongs.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

In addition, where features or aspects of the disclosure are described in terms of Markush groups, those skilled in the art will recognize that the disclosure is also thereby described in terms of any individual member or subgroup of members of the Markush group.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

1-49. (canceled)

50. A method of treating a Farnesoid X Receptor (FXR) mediated condition in a patient comprising administering to the patient a compound of Formula (I): 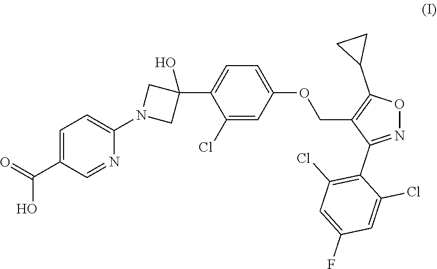 [SMILES: O=C(O)c1ccc(N2CC(O)(c3ccc(OCc4c(-c5c(Cl)cc(F)cc5Cl)noc4C4CC4)cc3Cl)C2)nc1]
or a pharmaceutically acceptable salt thereof.

51. The method of claim 1, wherein the FXR mediated condition is selected from the group consisting of:
a chronic intrahepatic or extrahepatic cholestatic condition;
liver fibrosis;
a chronic or obstructive inflammatory disorder of the liver;
liver cirrhosis;
liver steatosis or an associated syndrome;
a cholestatic or fibrotic effect that is associated with alcohol-induced cirrhosis or with a viral-borne form of hepatitis;
acute or chronic liver failure;
liver ischemia after major liver resection;
chemotherapy associated steatohepatitis (CASH);
Primary Sclerosing Cholangitis (PSC);
a neoplastic disease of the gastrointestinal tract or liver; and
an Inflammatory Bowel Disease (IBD);
a lipid disorder or lipoprotein disorder;
Type I Diabetes;
Type II Diabetes;
clinical complications of Type I and Type II Diabetes selected from the group consisting of diabetic nephropathy, diabetic neuropathy, diabetic retinopathy and other observed effects of clinically manifest long term Diabetes;
Non-Alcoholic Fatty Liver Disease (NAFLD);
Non-Alcoholic Steatohepatitis (NASH);
obesity;
a metabolic syndrome selected from the group consisting of combined conditions of dyslipidemia, diabetes and abnormally high body-mass index;
acute myocardial infarction;
acute stroke; and
thrombosis that occurs as an endpoint of chronic obstructive atherosclerosis;
a non-malignant hyperproliferative disorder;
a malignant hyperproliferative disorder selected from the group consisting of hepatocellular carcinoma, colon adenoma, and polyposis;
colon adenocarcinoma;
breast cancer;
pancreas adenocarcinoma; and
Barrett's esophagus.

52. A method of treating a Farnesoid X Receptor (FXR) mediated condition in a patient comprising administering to the patient a pharmaceutical composition comprising a therapeutically effective amount of an Apoptosis Signal-Regulating Kinase 1 (ASK1) inhibitor and a therapeutically effective amount of a compound having the structure of Formula (I): 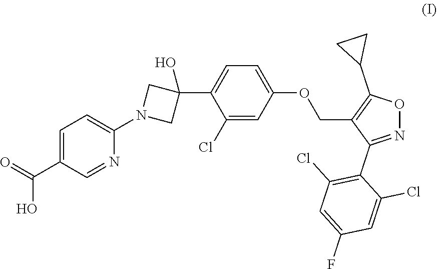 [SMILES: O=C(O)c1ccc(N2CC(O)(c3ccc(OCc4c(-c5c(Cl)cc(F)cc5Cl)noc4C4CC4)cc3Cl)C2)nc1]
or a pharmaceutically acceptable salt thereof.

53. The method of claim 52, wherein the ASK1 inhibitor is a compound of Formula (II): 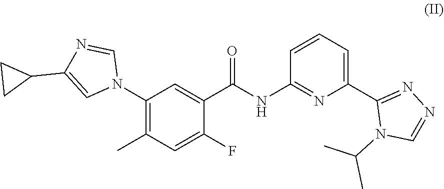 [SMILES: Cc1cc(F)c(C(=O)Nc2cccc(-c3nncn3C(C)C)n2)cc1-n1cnc(C2CC2)c1]
or a pharmaceutically acceptable salt, a stereoisomer, a mixture of stereoisomers, or a tautomer thereof.

54. A method of treating a Farnesoid X Receptor (FXR) mediated condition in a patient comprising administering to the patient a pharmaceutical composition comprising a therapeutically effective amount of an Acetyl CoA Carboxylase (ACC) inhibitor and a therapeutically effective amount of a compound having the structure of Formula (I): 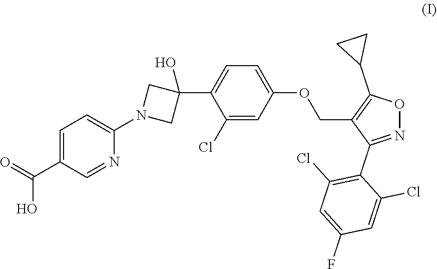 [SMILES: O=C(O)c1ccc(N2CC(O)(c3ccc(OCc4c(-c5c(Cl)cc(F)cc5Cl)noc4C4CC4)cc3Cl)C2)nc1]
or a pharmaceutically acceptable salt thereof.

55. The method of claim 54, wherein the ACC inhibitor is a compound of Formula (III): 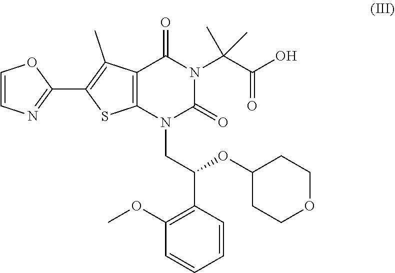 [SMILES: COc1ccccc1[C@H](Cn1c(=O)n(C(C)(C)C(=O)O)c(=O)c2c(C)c(-c3ncco3)sc21)OC1CCOCC1]
or a pharmaceutically acceptable salt, a stereoisomer, a mixture of stereoisomers, or a tautomer thereof.

56. A method of treating a Farnesoid X Receptor (FXR) mediated condition in a patient comprising administering to the patient a pharmaceutical composition comprising a therapeutically effective amount of a Thyroid Hormone Receptor (THR) β agonist and a therapeutically effective amount of a compound having the structure of Formula (I): 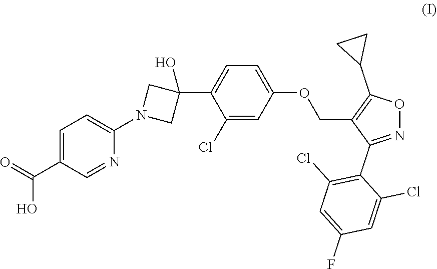 [SMILES: O=C(O)c1ccc(N2CC(O)(c3ccc(OCc4c(-c5c(Cl)cc(F)cc5Cl)noc4C4CC4)cc3Cl)C2)nc1]
or a pharmaceutically acceptable salt thereof.

57. The method of claim 56, wherein the THR β agonist is a compound of Formula (IV): 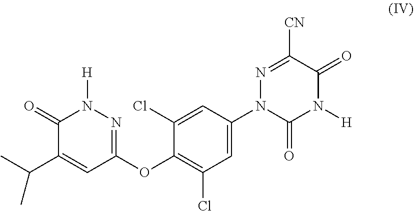 [SMILES: CC(C)c1cc(Oc2c(Cl)cc(-n3nc(C#N)c(=O)[nH]c3=O)cc2Cl)n[nH]c1=O]
or a pharmaceutically acceptable salt, a stereoisomer, a mixture of stereoisomers, or a tautomer thereof.

58. The method of claim 50, wherein the FXR mediated condition is Primary Biliary Cirrhosis (PBC).

59. The method of claim 50, wherein the FXR mediated condition is Primary Sclerosing Cholangitis (PSC).

60. The method of claim 50, wherein the FXR mediated condition is Non Alcoholic Steatohepatitis (NASH).

61. The method of claim 50, wherein the FXR mediated condition is Inflammatory Bowel Disease.

62. The method of claim 52, wherein the FXR mediated condition is Primary Biliary Cirrhosis (PBC).

63. The method of claim 52, wherein the FXR mediated condition is Primary Sclerosing Cholangitis (PSC).

64. The method of claim 52, wherein the FXR mediated condition is Non Alcoholic Steatohepatitis (NASH).

65. The method of claim 52, wherein the FXR mediated condition is Inflammatory Bowel Disease.

66. The method of claim 54, wherein the FXR mediated condition is Primary Biliary Cirrhosis (PBC).

67. The method of claim 54, wherein the FXR mediated condition is Primary Sclerosing Cholangitis (PSC).

68. The method of claim 54, wherein the FXR mediated condition is Non Alcoholic Steatohepatitis (NASH).

69. The method of claim 54, wherein the FXR mediated condition is Inflammatory Bowel Disease.

70. The method of claim 56, wherein the FXR mediated condition is Primary Biliary Cirrhosis (PBC).

71. The method of claim 56, wherein the FXR mediated condition is Primary Sclerosing Cholangitis (PSC).

72. The method of claim 56, wherein the FXR mediated condition is Non Alcoholic Steatohepatitis (NASH).

73. The method of claim 56, wherein the FXR mediated condition is Inflammatory Bowel Disease.


SEQUENCE LISTING (1 sequence)

SEQ ID NO: 1 - protein, 25 aa, synthetic construct
  REGION 1..25: note = Synthetic
CPSSHSSLTERHKILHRLLQEGSPS